(19) United States
(12) Patent Application Publication
(10) Pub. No.: US 2004/0132807 A1
(43) Pub. Date: Jul. 8, 2004

(54) Aurones as telomerase inhibitors

(76) Inventors: Dario Ballinari, Milanese (IT); Luisella Bonomini, Cesano Maderno (IT); Antonella Ermoli, Buccinasco (IT); Markus Gude, Laufelfingen (CH); Maria Menichincheri, Milan (IT); Jurgen Moll, Appiano Gentile (IT); Ermes Vanotti, Milan (IT)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/475,043

(22) PCT Filed: Apr. 15, 2002
(86) PCT No.: PCT/EP02/04191

(30) Foreign Application Priority Data
Apr. 18, 2001 (US) 09837846

(51) Int. Cl. A61K 31/343

(52) U.S. Cl. 514/469

(57) ABSTRACT

The present invention relates to know and novel substituted aurones active as telomerase inhibitors, to their use as therapeutic agents, in particular as antitumoral agents, to a process for their preparation as to pharmaceutical compositions comprising them.

### FIELD OF THE INVENTION

The present invention relates to methods for treating telomerase-modulated diseases, in particular cancer, to compounds that inhibit telomerase activity, to a process for their preparation, to their use as medicaments and to pharmaceutical compositions comprising them.

### BACKGROUND OF THE INVENTION

Cancer is one of the major causes of disease and the second leading cause of death in the western world. Most cancer patients still die due to metastatic disease. Despite the great increase in the knowledge and understanding of the regulatory mechanisms involved in the onset of malignancy, currently available treatments (including surgery, radiation and a variety of cytoreductive and hormone-based drugs, used alone or in combination, are still highly non specific and toxic to the patient, causing severe side effects including nausea and vomiting, hair loss, diarrhea, fatigue and ulcerations. These problems evidence the need for new and more effective anti-cancer therapies.

Recently an understanding of the mechanisms by which normal cells reach the state of replicative senescence, i.e. the loss of proliferative capacity that cells normally undergo in the cellular aging process has begun to emerge and in this respect telomerase appears to have a central role.

Telomerase is a ribonucleoprotein enzyme responsible in most eukaryotes for the complete replication and maintenance of chromosome ends, or telomeres, which are composed of repeated DNA sequences (in particular human telomeres are formed by 5′-TTAGGG repeats). Telomerase binds to telomeric DNA using as a template a sequence contained within the RNA component of the enzyme necessary for the addition of the short sequence repeats to the chromosome 3′ end (see Blackburn 1992, Annu. Rev. Biochem. 61, 113-129). In most human somatic cells telomerase activity cannot be detected and telomeres shorten with successive cell division: in fact actively dividing normal cells have the potential to lose 50-200 base pairs after each round of cell division, resulting in shortening of telomeres. Recently it has been hypothesized that the cumulative loss of telomeric DNA over repeated cell divisions may act as a trigger for cellular senescence and aging, and that regulation of telomerase may have important biological implications (see Harley 1991, Mutation Research, 256, 271-282). In fact in the absence of telomerase, telomeres shortening will eventually lead to cellular senescence by various mechanisms. This phenomenon, thought to be responsible for cellular aging, is termed the “mitotic clock” (see Holt et al. Nat. Biotechnol. 1996, 15, 1734-1741).

Telomerase activity is restored in immortalised cell lines and in more than 85% of human tumors, thus maintaining telomeres length stable (see Shay, J. W. and Bacchetti, S. Eur. J. Cancer, 1997, 33, 787-791). Thus in cancer cells having telomerase activity and where the malignant phenotype is due to the loss of cell cycle or growth controls or other genetic damage, telomeric DNA is not lost during cell division and telomers are maintained, thereby allowing the cancer cells to become immortal, leading to a terminal prognosis for the patient.

Telomerase inhibition can lead to telomere shortening in tumors and subsequent senescent phenotype (see Feng et al. Science, 1995, 269, 1236-1241). Moreover it has been recently shown (Hahn et al. Nature Med., 1999, 5, 1164-1170) that inhibition of telomerase activity by expressing in tumor cells a catalytically-inactive form of human TERT (TElomerase Reverse Transcriptase, the catalytic subunit of the enzyme) can cause telomere shortening and arrest of cell growth and apoptosis. In addition peptide-nucleic acids and 2′-O-MeRNA oligomers complementary to the template region of the RNA component of the enzyme have been reported to cause inhibition of telomerase activity, telomere shortening and cell death in certain tumor cell lines (see Herbert et al. PNAS, 1999, 96, 14276-14281; Shammas et al. Oncogene, 1999, 18, 6191-6200). These data support inhibition of telomerase activity as an innovative, selective and useful method for the development of new anticancer agents.

Thus compounds that inhibit telomerase activity can be used to treat cancer, as cancer cells express telomerase activity, while normal human somatic cells usually do not express telomerase activity at biologically relevant levels (i.e., at levels sufficient to maintain telomere length over many cell divisions). Also telomere length in tumors is reduced compared with non-transformed cells giving the possibility of a therapeutic window (see Nakamura et al. Cancer Letters 158, 2000, 179-184). Therefore a need exists to find molecules that inhibit the activity of telomerase and interfere with the growth of many types of cancer.

The present invention fulfills such a need by providing a highly general method of treating many—if not most—malignancies, as demonstrated by the highly varied human tumor cell lines and tumors having telomerase activity. Since the compounds of the present invention can be effective in providing treatments that discriminate between malignant and normal cells to a high degree, avoiding many of the deleterious side-effects present with most current chemotherapeutic regimes which rely on agents that kill dividing cells indiscriminately, they are also expected to exhibit greater safety and lack of toxic effects in comparison with traditional chemotherapeutic anticancer agents.

### SUMMARY OF THE INVENTION

The present invention discloses the function of substituted aurones active as telomerase inhibitors, their use as therapeutic agents, in particular as antitumoral agents, a process for their preparation, and pharmaceutical compositions comprising them.

These and other aspects of the invention are described in greater detail below.

### DETAILED DESCRIPTION OF THE INVENTION

It is an object of the present invention to provide a method for inhibiting telomerase enzyme, which comprises contacting said enzyme with an effective amount of a compound having the following formula (I)

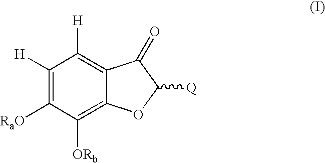

wherein:

each of Rₐ and R_{b} represents, independently, hydrogen, C₁-C₆ alkyl, C₁-C₆ alkylcarbonyl or, Rₐ and R_{b}, taken together, represent methylene;

[symbol] Q represents a group of formula (a), (b), (c), (d) or (e)

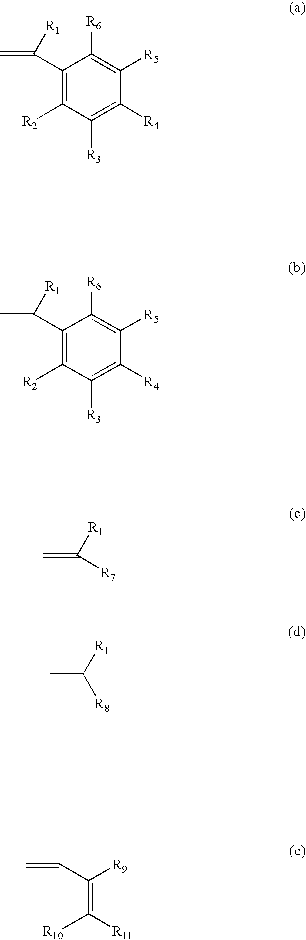

wherein:

in a group of formula (a)

R₁ represents hydrogen or C₁-C₆ alkyl;

each of R₂, R₅ and R₆ represents, independently, hydrogen, halogen, hydroxy, C₁-C₆ alkyl, haloalkyl, C₁-C₆ alkenyl, C₁-C₆ alkoxy, C₁-C₆ alkenyloxy, aryloxy, arylalkoxy, haloalkoxy, C₁-C₆ alkoxycarbonyl, carboxyl, nitro or cyano; and

each of R₃ and R₄ represents, independently, hydrogen, halogen, hydroxy, C₁-C₆ alkyl, haloalkyl, optionally substituted alkenyl, optionally substituted arylalkenyl, optionally substituted alkynyl, optionally substituted arylalkynyl, aryl, C₁-C₆ alkoxy, aryloxy, arylalkoxy, haloalkoxy, aminoalkoxy, carbalkoxy, C₁-C₆ alkoxycarbonylalkoxy, carboxyl, C₁-C₆ alkoxycarbonyl, acyloxy, amino, dialkylamino, optionally substituted dialkylamino, acylamino, thioalkyl, arylsulfonyl, alkylsulfonyl, arylsulfenyl, alkylsulfenyl, arylsulfanyl, alkylsulfanyl, nitro or cyano, or R₃ and R₄ taken together represent methylenedioxy;

in a group of formula (b)

R₁ represents hydrogen or C₁-C₆ alkyl;

each of R₂, R₅ and R₆ represents, independently, hydrogen, halogen, hydroxy, C₁-C₆ alkyl, haloalkyl, C₁-C₆ alkoxy, aryloxy, C₁-C₆ alkoxycarbonyl, carboxyl or cyano; and

each of R₃ and R₄ represents, independently, hydrogen, halogen, hydroxy, C₁-C₆ alkyl, aryl, C₁-C₆ alkoxy, aryloxy, aminoalkoxy, carbalkoxy, C₁-C₆ alkoxycarbonylalkoxy, carboxyl, C₁-C₆ alkoxycarbonyl, acyloxy, amino, dialkylamino, optionally substituted dialkylamino, acylamino or cyano, or R₃ and R₄, taken together, represent methylenedioxy;

in a group of formula (c)

R₁ represents hydrogen; and

R₇ represents a fused polycyclic optionally substituted aryl or a monocyclic, bicyclic or tricyclic optionally substituted heteroaryl;

in a group of formula (d)

R₁ represents hydrogen; and

R₈ represents a fused polycyclic optionally substituted aryl or a monocyclic, bicyclic or tricyclic optionally substituted heteroaryl; and

in a group of formula (e)

R₉ represents hydrogen, C₁-C₆ alkyl, halogen or optionally substituted aryl;

R₁₀ represents C₁-C₆ alkyl, C₁-C₄ alkoxy, carboxyl, alkoxycarbonyl, optionally substituted aryl or optionally substituted heteroaryl; and

R₁₁ represents hydrogen, halogen or optionally substituted aryl;

or any pharmaceutically acceptable salt of any of the foregoing.

It is a further object of the present invention to provide a method for treating a telomerase-modulated disease, which comprises administering to a mammal a therapeutic effective amount of a compound having the above formula (I) or a pharmaceutically acceptable salt thereof.

It is a still further object of the present invention to provide a method for treating a cancer disease related to abnormal cancer cell growth mediated by telomerase enzyme activity, which comprises administering to a mammal a therapeutic effective amount of a compound having the above formula (I) or a pharmaceutically acceptable salt thereof.

It is another object of the present invention to provide a method for treating a cancer, which comprises administering to a mammal a therapeutic effective amount of a compound having the above formula (I) or a pharmaceutically acceptable salt thereof.

According to still another aspect of the invention, a method is provided which involves the use of a compound having the above formula (I) in the preparation of a medicament. In particular embodiments, the medicament is for treating a proliferative disorder (e.g. a cancer). The present invention therefore also provides a compound having the above formula (I) for use in the preparation of a medicament having anticancer activity.

The present invention also comprises in its scope a pharmaceutical formulation for treating a telomerase-modulated disease, which comprises a compound having the above formula (I) or a pharmaceutically acceptable salt thereof and a pharmaceutically acceptable excipient.

The present invention also comprises in its scope a pharmaceutical formulation for treating a cancer disease related to abnormal cancer cell growth mediated by telomerase enzyme activity, which comprises a compound having the above formula (I) or a pharmaceutically acceptable salt thereof and a pharmaceutically acceptable excipient.

The present invention also comprises in its scope a pharmaceutical formulation for treating a cancer, which comprises a compound having the above formula (I) or a pharmaceutically acceptable salt thereof and a pharmaceutically acceptable excipient.

Some compounds of the aforementioned aurones of formula (I) and the pharmaceutically acceptable salt thereof are novel compounds and, as such, they represent a still another object of the present invention. Thus, the present invention includes in its scope also compounds of formula (IA), (IB), (IC), (ID) and (IE) as described below.

It is therefore an object of the present invention a compound of formula (IA) or a pharmaceutically acceptable salt thereof

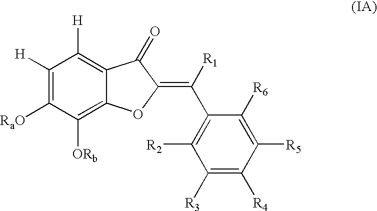

wherein

Rₐ and R_{b} are as defined in formula (I) above and

[symbol] Q is a group of formula (a) as defined in formula (I) above, provided that:

(i) when R₁ is hydrogen and Rₐ and R_{b} are at the same time methyl, then R₂, R₃, R₄, R₅, and R₆ are not at the same time hydrogen;

(ii) when R₁ is hydrogen, Rₐ and R_{b} are at the same time methyl, R₂, R₄, R₅ and R₆ are at the same time hydrogen, then R₃ is different from NO₂;

(iii) when R₁ is hydrogen, Rₐ and R_{b} are at the same time methyl, R₂, R₃, R₅ and R₆ are at the same time hydrogen, then R₄ is different from methoxy;

(iv) when R₁ is hydrogen, Rₐ and R_{b} are at the same time methyl, R₂, R₅ and R₆ are at the same time hydrogen, then R₃, R₄ are not at the same time methoxy or R₃ and R₄ taken together are not methylenedioxy;

(v) R₁, R₂, R₃, R₄, R₅, R₆, Rₐ and R_{b} are not at the same time hydrogen;

(vi) when R₁, Rₐ, R_{b}, R₃, R₄, R₅ and R₆ are at the same time hydrogen, then R₂ is different from Cl, NO₂ or OH;

(vii) when R₁, Rₐ, R_{b}, R₂, R₄, R₅ and R₆ are at the same time hydrogen, then R₃ is different from Cl, NO₂ or OH;

(viii) when R₁, Rₐ, R_{b}, R₂, R₃, R₅ and R₆ are at the same time hydrogen, then R₄ is different from Cl, NO₂ or OH;

(ix) when R₁, Rₐ, R_{b} R₂, R₅, and R₆ are at the same time hydrogen, then R₃ and R₄ are not at the same time methoxy or OH; or R₃ and R₄ taken together are not methylenedioxy;

(x) when R₁, Rₐ, R_{b} R₂, R₅ and R₆ are at the same time hydrogen, then R₃ is different from OH and R₄ is different from methoxy;

(xi) when R₁, Rₐ, R_{b}, R₂, R₅ and R₆ are at the same time hydrogen, then R₃ is different from methoxy and R₄ is different from OH;

(xii) when R₁, Rₐ, R_{b}, R₃, R₅ and R₆ are at the same time hydrogen, then R₂ and R₄ are not at the same time OH;

(xiii) when R₁, Rₐ, R_{b}, R₂ and R₆ are at the same time hydrogen, then R₃, R₄ and R₅ are not at the same time OH or methoxy;

(xiv) when R₁, Rₐ, R_{b}, R₂ and R₆ are at the same time hydrogen, then R₄ is different from OH and R₃ and R₅ are not at the same time methoxy;

(xv) when R₁, R₂, R₅ and R₆ are at the same time hydrogen, and Rₐ and R_{b} are at the same time acetyl, then R₃ and R₄ are not at the same time acetyloxy;

(xvi) when R₁, R₂, and R₆ are at the same time hydrogen, then R₃, R₄ and R₅ are not at the same time methoxy and acetyloxy;

(xvii) when R₁, Rₐ, R₂, R₅ and R₆ are at the same time hydrogen and R_{b} is methyl, then R₃ and R₄ are not at the same time methoxy or OH; and

(xviii) when R₁, R₂, R₅ and R₆ are at the same time hydrogen, Rₐ is acetyl and R_{b} is methyl, then R₃ and R₄ are not at the same time methoxy.

It is another object of the present invention a compound of formula (IB) or a pharmaceutically acceptable salt thereof

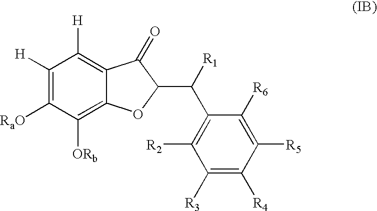

wherein

Rₐ and R_{b} are as defined in formula (I) above and

[symbol] Q is a group of formula (b) as defined in formula (I) above.

It is another object of the present invention a compound of formula (IC) or a pharmaceutically acceptable salt thereof

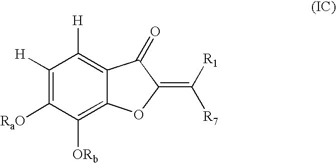

wherein

Rₐ and R_{b} are as defined in formula (I) above and

[symbol] Q is a group of formula (c) as defined in formula (I) above, provided that:

(i) when R₁ is hydrogen and R₇ is a group of formula

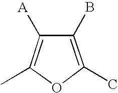

wherein A and B are at the same time hydrogen, then C is different from NO₂;

(ii) when R₁ is hydrogen and R₇ is a group of formula

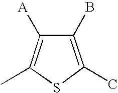

A, B and C are not at the same time hydrogen; and

(iii) when R₁ is hydrogen and R₇ is a group of formula

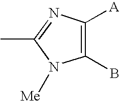

wherein A is hydrogen, then B is different from NO₂.

It is another object of the present invention a compound of formula (ID) or a pharmaceutically acceptable salt thereof

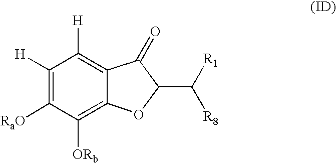

wherein

Rₐ and R_{b} are as defined in formula (I) above and

[symbol] Q is a group of formula (d) as defined in formula (I) above.

It is another object of the present invention a compound of formula (IE) or a pharmaceutically acceptable salt thereof

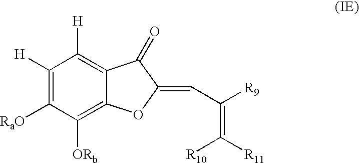

wherein

Rₐ and R_{b} are as defined in formula (I) above and

[symbol] Q is a group of formula (e) as defined in formula (I) above, provided that when R₉ and R₁₀ are at the same time hydrogen, then R₁₁ is different from unsubstituted phenyl.

The isolated double bond in formulae (I), (IA), (IC) and (IE) can present either (E) and (Z) stereochemistry, the most preferred being (Z).

Pharmaceutically acceptable salts of the compounds of formula (I), (IA), (IB), (IC), (ID) and (IE) are their salts with pharmaceutically acceptable either inorganic or organic acids such as, for instance, hydrochloric, hydrobromic, sulfuric, nitric, acetic, propionic, succinic, malonic, citric, tartaric, methanesulfonic and p-toluensulfonic acid, and their salts with pharmaceutically acceptable either inorganic or organic bases such as, for instance, hydroxides of alkali metals, for example, sodium or potassium, or alkaline earth metals such as, for instance, calcium, magnesium, zinc or aluminium, and organic bases, such as, for instance, aliphatic amines such as, for instance, methyl amine, diethylamine, dimethylamine, ethylamine or heterocyclic amines such as, for instance, piperidine. Such salts can be formed as known to those skilled in the art.

By the term “halogen” as used herein, is meant chlorine, bromine, iodine or fluorine.

By the term “alkyl” as used herein either alone or within other terms, is meant a saturated acyclic hydrocarbon including straight chain and branched chain groups. The alkyl group has, unless otherwise specified, 11 to 20 carbon atoms; preferably, it is a medium size alkyl having 1 to 6 carbon atoms; more preferably it is a lower alkyl having 1 to 4 carbon atoms. The alkyl group can be substituted or unsubstituted.

By the term “alkoxy” as used herein, is meant O-alkyl groups wherein the term “alkyl” is as defined above.

By the term “acyl” as used herein either alone or within other terms, is meant alkyl groups as defined above attached to a carbonyl group, i.e. alkyl-C═O groups, for instance formyl, acetyl, and pentanoyl.

C₁-C₆ alkyl is, preferably, C₁-C₄ alkyl, in particular methyl or ethyl.

C₁-C₆ acyl is, preferably, C₁-C₄ acyl, in particular acetyl or propanoyl.

C₁-C₆ alkoxy is, preferably, C₁-C₄ alkoxy, typically methoxy, ethoxy, propoxy or butoxy.

C₁-C₆ acyloxy is, preferably, C₁-C₄ acyloxy, preferably acetyloxy or propionyloxy.

C₁-C₄ acylamino is, preferably, acetylamino or propionylamino.

C₁-C₆ alkoxycarbonyl group is, preferably, a C₁-C₄ alkoxy-carbonyl group typically a C₁-C₂ one.

C₁-C₆ dialkylamino can be optionally substituted by cyano, halogen, acyloxy or alkoxycarbonyl.

By the term “aryl” as used herein, is meant an aromatic system having 20 or fewer carbon atoms, which can be a single ring or polycyclic aromatic rings fused or linked together as such that at least one part of the fused or linked rings forms the conjugated aromatic system. The aryl groups as defined immediately above, include but not limited to phenyl, naphthyl, anthryl, phenanthryl, fludrenyl and pyrenyl.

By the term “heteroaryl” as used herein, is meant aromatic heterocyclic groups containing one or more heteroatoms each selected from 0, S and N, wherein each heterocyclic group has from 5-10 atoms in its ring system. Examples of aromatic heterocyclic groups are thiophenyl, pyrazolyl, furyl, thiazolyl, isoxazolyl, oxazolyl, triazolyl, pyrrolyl, pyrazinyl, imidazolyl, pyridinyl, pyridinyl N-oxides, pyrimidinyl, 2,4-dioxo-1,2,3,4-tetrahydro-5-pyrimidinyl, benzothiophenyl, benzoxazolyl, benzotriazolyl, benzofuranyl, benzoimidazolyl, indolyl, quinolinyl, indazolyl, 2,3-dihydro-1,4-benzodioxinyl, chromenyl-4-ones, chromenyl and carbazolyl.

The aryl and heteroaryl groups as just defined above can be optionally substituted by from one to four substituents from the group including halogen, cyano, hydroxy, nitro, amino, C₁-C₆ monoalkylamino, C₁-C₆ dialkylamino, C₁-C₆ alkyl, cycloalkyl, C₁-C₆ alkylaryl, alkenyl, alkynyl, aryl, 5-10 membered heterocyclyl, alkoxy, aryloxy, C₁-C₆ alkylthio, arylthio, C₁-C₆ alkylsulfonyl, arylsulfonyl, C₁-C₆ acyl, aroyl, C₁-C₆ acyloxy, C₁-C₄ acylamino, C₁-C₆ alkoxycarbonyl, aryloxycarbonyl, carboxyl, C₁-C₆ alkylsulfonylamino, arylsulfonylamino, C₁-C₆ alkylaminosulfonyl and arylaminosulfonyl.

By the term “cycloalkyl” as used herein, is meant a C₁-C₁₀ do all-carbon monocyclic or fused ring, including, e.g., cyclopropane, cyclobutane, cyclopentane, cyclohexane and cycloheptane.

By the term “alkenyl” as used herein, is meant an alkyl group, as defined herein, consisting of at least two carbon atoms and at least one carbon-carbon double bond. The alkenyl group as just defined above can be optionally substituted by carboxy, aryl, phenyl, alkoxycarbonyl.

By the term “alkynyl” as used herein, is meant an alkyl group, as defined herein, consisting of at least two carbon atoms and at least one carbon-carbon triple bond. The alkynyl group as just defined above can be optionally substituted by aryl.

By the term “aroyl” as used herein, is meant aryl groups, as defined above, attached to a carbonyl group, i.e. aryl-C═O, for instance benzoyl and toluoyl.

By the term haloalkyl as used herein, is meant an alkyl bearing one or more halogens, being alkyl and halogen as defined above.

By the term haloalkoxy as used herein, is meant an alkoxy bearing one or more halogens, being alkoxy and halogen as defined above.

By the term aminoalkoxy as used herein, is meant an alkoxy bearing one or more amino groups, being alkoxy as defined above.

By the term alkoxycarbonylalkoxy as used herein, is meant a group alkyl-O—CO-alkyl-O—, being alkyl as defined above.

By the term arylalkoxy as used herein, is meant an aryl linked to the alkylic chain of the alkoxy, being alkoxy as defined above.

By the term alkenyloxy as used herein, is meant an alkoxy group, as defined herein, consisting of at least two carbons and at least one carbon-carbon double bond.

The term “sulfonyl”, whether used alone or linked to other terms such as, for instance, alkylsulfonyl or arylsulfonyl, denotes respectively divalent radicals —SO₂—. The term “alkylsulfonyl”, embraces alkyl radicals attached to a sulfonyl radical, where alkyl is as defined above. The term “arylsulfonyl”, embraces aryl radicals attached to a sulfonyl radical, where aryl is as defined above.

The term “sulfenyl” whether used alone or linked to other terms such as, for instance, alkylsulfenyl or arylsulfenyl, denotes respectively divalent radicals —S—. The term “alkylsulfenyl”, embraces alkyl radicals attached to a sulfenyl radical, where alkyl is as defined above. The term “arylsulfenyl”, embraces aryl radicals attached to a sulfenyl radical, where aryl is as defined above.

The term “sulfanyl” whether used alone or linked to other terms such as, for instance, alkylsulfanyl or arylsulfanyl, denotes respectively divalent radicals —SO—. The term “alkylsulfanyl”, embraces alkyl radicals attached to a sulfanyl radical, where alkyl is as defined above. The term “arylsulfanyl”, embraces aryl radicals attached to a sulfanyl radical, where aryl is as defined above.

By the term “carbalkoxy” as used herein, is meant HOOC-alkyl-O— group, that is an alkoxy bearing a carboxy on the alkyl chain, wherein “alkoxy” and “alkyl” are as defined above.

The terms “malignant neoplasm”, “cancer”, “tumor” and “solid tumor cancer” are used interchangeably herein to refer to the condition well known to those skilled in the art as the life-threatening disease commonly referred to simply as “cancer”. The term “cancer” as used herein, is meant a disease characterized by excessive, uncontrolled growth of abnormal cells, which invades and destroys other tissues and includes all human cancers such as carcinomas, sarcomas, leukemias and lymphomas. For example, the term “cancer” comprises prostate, breast, lung, colorectal, bladder, uterine, skin, kidney, pancreatic, ovarian, liver and stomach cancer.

By the term “chemotherapeutic agent” as used herein, is meant a chemical substance or drug used to treat a disease; the term is most often applied to such substances or drugs which are used primarily for the treatment of cancer.

By the term “treating” as used herein, is meant reversing, alleviating, ameliorating, limiting, inhibiting the progress of, or preventing the disorder or condition to which such term applies, or one or more symptoms of such disorder or condition. The term “treatment” as used herein, refers to the act of treating as “treating” is defined immediately above.

By the term “method” as used herein, is meant manners, means, techniques and procedures for accomplishing a given task including, but not limited to, those manners, means, techniques and procedures either known to, or readily developed from known manners, means, techniques and procedures by, practitioners of the chemical, pharmacological, biological, biochemical and medical arts.

By the term “administered” or “administering” as used herein, is meant standard delivery methods, e.g, parenteral administration, including continuous infusion and intravenous, intramuscular and subcutaneous injections, and oral administration.

The term “modulated” as used herein includes governed, controlled, provoked and induced.

By the term “coordinated” as used herein includes simultaneous, separate and/or sequential.

By the term “mammal” as used herein, is meant any of a class of warm-blooded higher vertebrates, that nourish their young with milk secreted by mammary glands, have the skin usually more or less covered with hair, and include humans.

By the term “physiologically acceptable carrier” used herein, is meant a carrier or diluent that does not cause significant irritation to an organism and does not abrogate the biological activity and properties of the administered compound.

By the term “excipient” as used herein, is meant an inert substance added to a pharmaceutical composition to further facilitate administration of a compound.

By the term “disease” as used herein, is meant a kind or instance of impairment of a living being that interferes with normal bodily function.

The compounds of this invention can contain an asymmetric carbon atom and some of the compounds of this invention can contain one or more asymmetric centers and can thus give rise to optical isomers and diastereomers. While shown without respect to stereochemistry in formula (I), the present invention includes such optical isomers and diastereomers; as well as the racemic and resolved, enantiomerically pure R and S stereoisomers; as well as other mixtures of the R and S stereoisomers and pharmaceutically acceptable salts thereof.

Some of the compounds described herein can contain one or more ketonic or aldehydic carbonyl groups or combinations thereof alone or as part of a heterocyclic ring system. Such carbonyl groups can exist in part or principally in the “keto” form and in part or principally as one or more “enol” forms of each aldehyde and ketone group present. Compounds of the present invention having aldehydic or ketonic carbonyl groups are meant to include both “keto” and “enol” tautomeric forms. Some of the compounds described herein can contain one or more imine or enamine groups or combinations thereof. Such groups can exist in part or principally in the “imine” form and in part or principally as one or more “enamine” forms of each group present. Compounds of the present invention having said imine or enamine groups are meant to include both “imine” and “enamine” tautomeric forms.

It is therefore understood that the present invention includes in its scope all the possible tautomeric forms of the compounds of formula (I).

The present invention also includes within its scope pharmaceutically acceptable bio-precursors (otherwise known as pro-drugs) of the compounds of formula (I), i.e. compounds which have a different formula (I) above, but which nevertheless upon administration to a human being are converted directly or indirectly in vivo into a compound of formula (I).

A further object of the present invention is to provide a pharmaceutical composition, which comprises as an active principle a compound of formula (IA), (IB), (IC), (ID) or (IE) as defined above or a pharmaceutically acceptable salt thereof and a pharmaceutically acceptable excipient.

The compounds of formula (IA), (IB), (IC), (ID) or (IE) represent selected classes of compounds of formula (I) and are thus also effective as telomerase inhibitors and active in the treatment of all the diseases for which the compounds of formula (I) have been indicated as therapeutic agents. A compound of formula (IA), (IB), (IC), (ID) or (IE) as defined above or a pharmaceutically acceptable salt thereof for use as a medicament, in particular for the treatment of a telomerase-modulated disease, more in particular for the treatment of a cancer disease related to abnormal cancer cell growth mediated by telomerase enzyme activity, is therefore encompassed by the scope of the present invention.

Examples of specific compounds of the invention include:

2-(3,4-dihydroxybenzylidene)-6,7-dihydroxy-1-benzofuran-3(2H)-one (compound 1);

2-(1,3-benzodioxol-5-ylmethylene)-6,7-dihydroxy-1-benzofuran-3(2H)-one (compound 2);

2-(3,4-dimethoxybenzylidene)-6,7-dihydroxy-1-benzofuran-3(2H)-one (compound 3);

2-(2,4-dihydroxybenzylidene)-6,7-dihydroxy-1-benzofuran-3(2H)-one (compound 4);

6,7-dimethoxy-2-(3-nitrobenzylidene)-1-benzofuran-3(2H)-one;

6,7-dimethoxy-2-(4-methoxybenzylidene)-1-benzofuran-3(2H)-one;

2-(1,3-benzodioxol-5-ylmethylene)-6,7-dimethoxy-1-benzofuran-3(2H)-one;

2-(3,4-dimethoxybenzylidene)-6,7-dimethoxy-1-benzofuran-3(2H)-one;

2-benzylidene-6,7-dihydroxy-1-benzofuran-3(2H)-one;

2-(4-chlorobenzylidene)-6,7-dihydroxy-1-benzofuran-3(2H)-one;

2-(3-chlorobenzylidene)-6,7-dihydroxy-1-benzofuran-3(2H)-one;

2-(2-chlorobenzylidene)-6,7-dihydroxy-1-benzofuran-3(2H)-one;

6,7-dihydroxy-2-(4-hydroxybenzylidene)-1-benzofuran-3(2H)-one;

6,7-dihydroxy-2-(3-hydroxybenzylidene)-1-benzofuran-3(2H)-one;

6,7-dihydroxy-2-(2-hydroxybenzylidene)-1-benzofuran-3(2H)-one;

6,7-dihydroxy-2-(4-nitrobenzylidene)-1-benzofuran-3(2H)-one;

6,7-dihydroxy-2-(3-nitrobenzylidene)-1-benzofuran-3(2H)-one;

6,7-dihydroxy-2-(2-nitrobenzylidene)-1-benzofuran-3(2H)-one;

6,7-dihydroxy-2-(3-hydroxy-4-methoxybenzylidene)-1-benzofuran-3(2H)-one;

6,7-dihydroxy-2-(4-hydroxy-3-methoxybenzylidene)-1-benzofuran-3(2H)-one;

6,7-dihydroxy-2-(3,4,5-trihydroxybenzylidene)-1-benzofuran-3(2H)-one;

6,7-dihydroxy-2-(3,4,5-trimethoxybenzylidene)-1-benzofuran-3(2H)-one;

6,7-dihydroxy-2-(4-hydroxy-3,5-dimethoxybenzylidene)-1-benzofuran-3(2H)-one;

6-(acetyloxy)-2-[3,4-bis(acetyloxy)benzylidene]-3-oxo-1-benzofuran-7(3H)-yl acetate;

6-(acetyloxy)-3-oxo-2-(3,4,5-trimethoxybenzylidene)-2,3-dihydro-1-benzofuran-7-yl acetate;

6-(acetyloxy)-3-oxo-2-[3,4,5-tris(acetyloxy)benzylidene]-2,3-dihydro-1-benzofuran-7-yl acetate;

2-(3,4-dimethoxybenzylidene)-6-hydroxy-7-methoxy-1-benzofuran-3(2H)-one;

2-(3,4-dihydroxybenzylidene)-6-hydroxy-7-methoxy-1-benzofuran-3(2H)-one;

2-(3,4-dimethoxybenzylidene)-7-methoxy-3-oxo-2,3-dihydro-1-benzofuran-6-yl acetate;

2-[1-(4-hydroxyphenyl)ethylidene]-6-methoxy-1-benzofuran-3(2H)-one;

4-[1-(6-methoxy-3-oxo-1-benzofuran-2(3H)-ylidene)ethyl]phenyl acetate;

2-[1-(3,4-dihydroxyphenyl)ethylidene]-4,6-dihydroxy-1-benzofuran-3(2H)-one;

6,7-dihydroxy-2-[(5-nitro-2-furyl)methylene]-1-benzofuran-3(2H)-one;

6,7-dimethoxy-2-[(5-nitro-2-furyl)methylene]-1-benzofuran-3(2H)-one;

6-(acetyloxy)-2-[(5-nitro-2-furyl)methylene]-3-oxo-2,3-dihydro-1-benzofuran-7-yl acetate;

6,7-dihydroxy-2-(2-thienylmethylene)-1-benzofuran-3(2H)-one;

6,7-dimethoxy-2-(2-thienylmethylene)-1-benzofuran-3(2H)-one;

6,7-dihydroxy-2-[(1-methyl-5-nitro-1H-imidazol-2-yl)methylene]-1-benzofuran-3(2H)-one;

6,7-dimethoxy-2-[(1-methyl-5-nitro-1H-imidazol-2-yl)methylene]-1-benzofuran-3(2H)-one;

6,7-dihydroxy-2-[3-phenyl-2-propenylidene]-1-benzofuran-3(2H)-one;

6-(acetyloxy)-3-oxo-2-[3-phenyl-2-propenylidene]-2,3-dihydro-1-benzofuran-7-yl acetate;

and the pharmaceutically acceptable salt thereof.

Additional examples of novel compounds according to the invention include compounds listed under Group 1, 2 and 3.

### Group 1 (Compound 5-Compound 89)

2-(3,4-dichlorobenzylidene)-6,7-dihydroxy-1-benzofuran-3(2H)-one (compound 5);

2-(3,4-dihydroxybenzylidene)-6,7-dimethoxy-1-benzofuran-3(2H)-one (compound 6);

2-[1-(3,4-dimethoxyphenyl)ethylidene]-6,7-dihydroxy-1-benzofuran-3(2H)-one (compound 7);

2-(2,5-dihydroxybenzylidene)-6,7-dihydroxy-1-benzofuran-3(2H)-one (compound 8);

2-(3-fluoro-2-hydroxybenzylidene)-6,7-dihydroxy-1-benzofuran-3(2H)-one (compound 9);

6,7-dihydroxy-2-[(7-methoxy-1,3-benzodioxol-5-yl)methylene]-1-benzofuran-3(2H)-one (compound 10);

6,7-dihydroxy-2-(2,4,6-trifluorobenzylidene)-1-benzofuran-3(2H)-one (compound 11);

6,7-dihydroxy-2-(2-hydroxy-3-methoxybenzylidene)-1-benzofuran-3(2H)-one (compound 12);

2-(3,5-dimethylbenzylidene)-6,7-dihydroxy-1-benzofuran-3(2H)-one (compound 13);

2-(3,4,5-trihydroxybenzylidene)-6,7-dihydroxy-1-benzofuran-3(2H)-one (compound 14);

2-(4-chloro-3-fluorobenzylidene)-6,7-dihydroxy-1-benzofuran-3(2H)-one (compound 15);

2-[4-(benzyloxy)benzylidene]-6,7-dihydroxy-1-benzofuran-3(2H)-one (compound 16);

5-[(6,7-dihydroxy-3-oxo-1-benzofuran-2(3H)-ylidene)methyl]-2-hydroxybenzoic acid (compound 17);

2-(5-bromo-2-hydroxybenzylidene)-6,7-dihydroxy-1-benzofuran-3(2H)-one (compound 18);

3-[(6,7-dihydroxy-3-oxo-1-benzofuran-2(3H)-ylidene)methyl]benzoic acid (compound 19);

6,7-dihydroxy-2-[4-(phenylethynyl)benzylidene]-1-benzofuran-3(2H)-one (compound 20);

2-(3,5-ditert-butyl-2-hydroxybenzylidene)-6,7-dihydroxy-1-benzofuran-3(2H)-one (compound 21);

2-(3,5-dihydroxybenzylidene)-6,7-dihydroxy-1-benzofuran-3(2H)-one (compound 22);

3-{4-[(6,7-dihydroxy-3-oxo-1-benzofuran-2(3H)-ylidene)methyl]phenyl}-2-propenoic acid (compound 23);

2-(3,4-dihydroxy-5-methoxybenzylidene)-6,7-dihydroxy-1-benzofuran-3(2H)-one (compound 24); 2-[2-fluoro-4-(trifluoromethyl)benzylidene]-6,7-dihydroxy-1-benzofuran-3(2H)-one (compound 25);

6,7-dihydroxy-2-(3,4-dimethylbenzylidene)-1-benzofuran-3(2H)-one (compound 26);

2-[3-fluoro-4-(trifluoromethyl)benzylidene]-6,7-dihydroxy-1-benzofuran-3(2H)-one (compound 27);

2-(3-bromo-5-chloro-2-hydroxybenzylidene)-6,7-dihydroxy-1-benzofuran-3(2H)-one (compound 28);

2-[4-(dimethylamino)-2-methoxybenzylidene]-6,7-dihydroxy-1-benzofuran-3(2H)-one (compound 29);

2-[4-(benzyloxy)-2-hydroxybenzylidene]-6,7-dihydroxy-1-benzofuran-3(2H)-one (compound 30);

2-[4-(benzyloxy)-2-methoxybenzylidene]-6,7-dihydroxy-1-benzofuran-3(2H)-one (compound 31);

2-(2-fluoro-4-chlorobenzylidene)-6,7-dihydroxy-1-benzofuran-3(2H)-one (compound 32);

2-[2-(difluoromethoxy)benzylidene]-6,7-dihydroxy-1-benzofuran-3(2H)-one (compound 33);

6,7-dihydroxy-2-(2-vinylbenzylidene)-1-benzofuran-3(2H)-one (compound 34);

methyl 2-[(6,7-dihydroxy-3-oxo-1-benzofuran-2(3H)-ylidene)methyl]-3,5-dimethoxybenzoate (compound 35);

2-[(6,7-dihydroxy-3-oxo-1-benzofuran-2(3H)-ylidene)methyl]benzonitrile (compound 36);

2-(2,3-dichlorobenzylidene)-6,7-dihydroxy-1-benzofuran-3(2H)-one (compound 37);

2-[4-(diethylamino)benzylidene]-6,7-dihydroxy-1-benzofuran-3(2H)-one (compound 38);

2-(2,4-dimethoxy-3-methylbenzylidene)-6,7-dihydroxy-1-benzofuran-3(2H)-one (compound 39);

6,7-dihydroxy-2-(2,3,4,5,6-pentamethylbenzylidene)-1-benzofuran-3(2H)-one (compound 40);

2-(2-bromo-4,5-dimethoxybenzylidene)-6,7-dihydroxy-1-benzofuran-3(2H)-one (compound 41);

2-(3,5-dimethoxybenzylidene)-6,7-dihydroxy-1-benzofuran-3(2H)-one (compound 42);

4-[(6,7-dihydroxy-3-oxo-1-benzofuran-2(3H)-ylidene)methyl]-2,6-dimethoxyphenyl acetate (compound 43);

2-(3-ethoxy-4-methoxybenzylidene)-6,7-dihydroxy-1-benzofuran-3(2H)-one (compound 44);

2-(2,4-difluorobenzylidene)-6,7-dihydroxy-1-benzofuran-3(2H)-one (compound 45);

2-(2,5-difluorobenzylidene)-6,7-dihydroxy-1-benzofuran-3(2H)-one (compound 46);

2-(2,6-difluorobenzylidene)-6,7-dihydroxy-1-benzofuran-3(2H)-one (compound 47);

2-(4-butoxybenzylidene)-6,7-dihydroxy-1-benzofuran-3(2H)-one (compound 48);

2-(3-chloro-4-fluorobenzylidene)-6,7-dihydroxy-1-benzofuran-3(2H)-one (compound 49);

2-(2,3,6-trichlorobenzylidene)-6,7-dihydroxy-1-benzofuran-3(2H)-one (compound 50);

2-(3,5-difluorobenzylidene)-6,7-dihydroxy-1-benzofuran-3(2H)-one (compound 51);

2-(2,3-difluorobenzylidene)-6,7-dihydroxy-1-benzofuran-3(2H)-one (compound 52);

2-(2,3,5-trichlorobenzylidene)-6,7-dihydroxy-1-benzofuran-3(2H)-one (compound 53);

2-(5-bromo-2,4-dimethoxybenzylidene)-6,7-dihydroxy-1-benzofuran-3(2H)-one (compound 54);

2-(2,6-dimethoxybenzylidene)-6,7-dihydroxy-1-benzofuran-3(2H)-one (compound 55);

2-[4-(hexyloxy)benzylidene]-6,7-dihydroxy-1-benzofuran-3(2H)-one (compound 56);

2-(3-methyl-4-methoxybenzylidene)-6,7-dihydroxy-1-benzofuran-3(2H)-one (compound 57);

4-[(6,7-dihydroxy-3-oxo-1-benzofuran-2(3H)-ylidene)methyl]phenyl acetate (compound 58);

6,7-dihydroxy-2-(4-propoxybenzylidene)-1-benzofuran-3(2H)-one (compound 59);

2-(1,3-benzodioxol-4-ylmethylene)-6,7-dihydroxy-1-benzofuran-3(2H)-one (compound 60);

6,7-dihydroxy-2-(4-phenoxybenzylidene)-1-benzofuran-3(2H)-one (compound 61);

2-[4-(benzyloxy)-3-methoxybenzylidene]-6,7-dihydroxy-1-benzofuran-3(2H)-one (compound 62);

2-(2-chloro-6-fluorobenzylidene)-6,7-dihydroxy-1-benzofuran-3(2H)-one (compound 63);

2-(2,3-dimethyl-4-methoxybenzylidene)-6,7-dihydroxy-1-benzofuran-3(2H)-one (compound 64);

2-(2,5-dimethyl-4-methoxybenzylidene)-6,7-dihydroxy-1-benzofuran-3(2H)-one (compound 65);

6,7-dihydroxy-2-(2,3,4,5,6-pentafluorobenzylidene)-1-benzofuran-3(2H)-one (compound 66);

6,7-dihydroxy-2-(3-phenoxybenzylidene)-1-benzofuran-3(2H)-one (compound 67);

2-[3-(4-chlorophenoxy)benzylidene]-6,7-dihydroxy-1-benzofuran-3(2H)-one (compound 68);

6,7-dihydroxy-2-[3-(4-methoxyphenoxy)benzylidene]-1-benzofuran-3(2H)-one (compound 69);

6,7-dihydroxy-2-[3-(4-methylphenoxy)benzylidene]-1-benzofuran-3(2H)-one (compound 70);

2-{4-[3-(dimethylamino)propoxy]benzylidene}-6,7-dihydroxy-1-benzofuran-3(2H)-one (compound 71);

2-(2-fluoro-4-bromobenzylidene)-6,7-dihydroxy-1-benzofuran-3(2H)-one (compound 72);

2-(2,4-diethoxy-3-methylbenzylidene)-6,7-dihydroxy-1-benzofuran-3(2H)-one (compound 73);

2-[2-chloro-5-(trifluoromethyl)benzylidene]-6,7-dihydroxy-1-benzofuran-3(2H)-one (compound 74);

2-[4-fluoro-2-(trifluoromethyl)benzylidene]-6,7-dihydroxy-1-benzofuran-3(2H)-one (compound 75);

2-[2-fluoro-6-(trifluoromethyl)benzylidene]-6,7-dihydroxy-1-benzofuran-3(2H)-one (compound 76);

2-(4-tert-butylbenzylidene)-6,7-dihydroxy-1-benzofuran-3(2H)-one (compound 77);

6,7-dihydroxy-2-(2,3,5,6-tetrafluorobenzylidene)-1-benzofuran-3(2H)-one (compound 78);

6,7-dihydroxy-2-[4-(trifluoromethoxy)benzylidene]-1-benzofuran-3(2H)-one (compound 79);

2-[4-(dibutylamino)benzylidene]-6,7-dihydroxy-1-benzofuran-3(2H)-one (compound 80);

2-{4-[bis(2-cyanoethyl)amino]benzylidene}-6,7-dihydroxy-1-benzofuran-3(2H)-one (compound 81);

6,7-dihydroxy-2-[3-(trifluoromethoxy)benzylidene]-1-benzofuran-3(2H)-one (compound 82);

2-(2-chloro-4-fluorobenzylidene)-6,7-dihydroxy-1-benzofuran-3(2H)-one (compound 83);

2-(2-methyl-3-fluorobenzylidene)-6,7-dihydroxy-1-benzofuran-3(2H)-one (compound 84);

2-[2-fluoro-3-(trifluoromethyl)benzylidene]-6,7-dihydroxy-1-benzofuran-3(2H)-one (compound 85);

2-[4-(difluoromethoxy)benzylidene]-6,7-dihydroxy-1-benzofuran-3(2H)-one (compound 86);

2-[2,5-bis(trifluoromethyl)benzylidene]-6,7-dihydroxy-1-benzofuran-3(2H)-one (compound 87);

2-[4-fluoro-3-(trifluoromethyl)benzylidene]-6,7-dihydroxy-1-benzofuran-3(2H)-one (compound 88);

2-(3,4-dihydroxybenzyl)-6,7-dihydroxy-1-benzofuran-3(2H)-one (compound 89);

Group 2 (Compound 90-Compound 120)

6,7-dihydroxy-2-(3-pyridinylmethylene)-1-benzofuran-3(2H)-one (compound 90);

6,7-dihydroxy-2-[(6-hydroxy-4H-chromen-3-yl)methylene]-1-benzofuran-3(2H)-one (compound 91);

6,7-dihydroxy-2-[(6-methoxy-2-naphthyl)methylene]-1-benzofuran-3(2H)-one (compound 92);

6,7-dihydroxy-2-[(5-methyl-2-thienyl)methylene]-1-benzofuran-3(2H)-one (compound 93);

6,7-dihydroxy-2-[(5-methoxy-1H-indol-2-yl)methylene]-1-benzofuran-3(2H)-one (compound 94);

6,7-dihydroxy-2-[(1-methyl-1H-benzimidazol-2-yl)methylene]-1-benzofuran-3(2H)-one (compound 95);

2-[(1-acetyl-1H-indol-3-yl)methylene]-6,7-dihydroxy-1-benzofuran-3(2H)-one (compound 96);

6,7-dihydroxy-2-[(4-methyl-1H-imidazol-5-yl)methylene]-1-benzofuran-3(2H)-one (compound 97);

5-[(6,7-dihydroxy-3-oxo-1-benzofuran-2(3H)-ylidene)methyl]-2,4(1H,3H)-pyrimidinedione (compound 98);

6,7-dihydroxy-2-[(1-methyl-1H-imidazol-2-yl)methylene]-1-benzofuran-3(2H)-one (compound 99);

6,7-dihydroxy-2-(1H-indol-7-ylmethylene)-1-benzofuran-3(2H)-one (compound 100);

6,7-dihydroxy-2-[(3-methyl-1-benzothien-2-yl)methylene]-1-benzofuran-3(2H)-one (compound 101);

2-(2,3-dihydro-1,4-benzodioxin-6-ylmethylene)-6,7-dihydroxy-1-benzofuran-3(2H)-one (compound 102);

2-(9-anthrylmethylene)-6,7-dihydroxy-1-benzofuran-3(2H)-one (compound 103);

6,7-dihydroxy-2-(1-pyrenylmethylene)-1-benzofuran-3(2H)-one (compound 104);

{5-[(6,7-dihydroxy-3-oxo-1-benzofuran-2(3H)-ylidene)methyl]-2-furyl}methyl acetate (compound 105);

6,7-dihydroxy-2-(9-phenanthrylmethylene)-1-benzofuran-3(2H)-one (compound 106);

2-(9H-fluoren-2-ylmethylene)-6,7-dihydroxy-1-benzbfuran-3(2H)-one (compound 107);

2-[(10-chloro-9-anthryl)methylene]-6,7-dihydroxy-1-benzofuran-3(2H)-one (compound 108);

2-[(10-methyl-9-anthryl)methylene]-6,7-dihydroxy-1-benzofuran-3(2H)-one (compound 109);

6,7-dihydroxy-2-({5-[2-(trifluoromethyl)phenyl]-2-furyl}methylene)-1-benzofuran-3(2H)-one (compound 110);

2-{[5-(2-chlorophenyl)-2-furyl]methylene}-6,7-dihydroxy-1-benzofuran-3(2H)-one (compound 111);

2-[(4,5-dimethyl-2-furyl)methylene]-6,7-dihydroxy-1-benzofuran-3(2H)-one (compound 112);

2-[(5-bromo-2-furyl)methylene]-6,7-dihydroxy-1-benzofuran-3(2H)-one (compound 113);

2-{[5-(3-chlorophenyl)-2-furyl]methylene}-6,7-dihydroxy-1-benzofuran-3(2H)-one (compound 114);

6,7-dihydroxy-2-{[1-(phenylsulfonyl)-1H-pyrol-2-yl]methylene}-1-benzofuran-3(2H)-one (compound 115);

6,7-dihydroxy-2-({5-[3-(trifluoromethyl)phenyl]-2-furyl}methylene)-1-benzofuran-3(2H)-one (compound 116);

2-[(5-ethyl-2-furyl)methylene]-6,7-dihydroxy-1-benzofuran-3(2H)-one (compound 117);

6,7-dihydroxy-2-[(5-chloro-2-thienyl)methylene]-1-benzofuran-3(2H)-one (compound 118);

2-[(5-chloro-3-methyl-1-phenyl-1H-pyrazol-4-yl)methylene]-6,7-dihydroxy-1-benzofuran-3(2H)-one (compound 119);

2-[(2,4-dimethoxy-5-pyrimidinyl)methylene]-6,7-dihydroxy-1-benzofuran-3(2H)-one (compound 120); and the pharmaceutically acceptable salt thereof.

### Group 3 (Compound 121-Compound 125)

6,7-dihydroxy-2-[3-(4-hydroxy-3-methoxyphenyl)-2-propenylidene]-1-benzofuran-3(2H)-one (compound 121);

2-(3,3-diphenyl-2-propenylidene)-6,7-dihydroxy-1-benzofuran-3(2H)-one (compound 122);

6,7-dihydroxy-2-[2-methyl-3-phenyl-2-propenylidene]-1-benzofuran-3(2H)-one (compound 123);

2-{3-[4-(dimethylamino)phenyl]-2-propenylidene}-6,7-dihydroxy-1-benzofuran-3(2H)-one (compound 124);

2-[3-(4-tert-butylphenyl)-2-methyl-2-propenylidene]-6,7-dihydroxy-1-benzofuran-3(2H)-one (compound 125); the pharmaceutically acceptable salt thereof.

Further examples of compounds of the invention include:

6,7-dihydroxy-2-(1-phenylethylidene)-1-benzofuran-3(2H)-one (compound 126);

6,7-dihydroxy-2-[1-(4-hydroxyphenyl)ethylidene]-1-benzofuran-3(2H)-one (compound 127);

6,7-dihydroxy-2-[1-(3-hydroxyphenyl)ethylidene]-1-benzofuran-3(2H)-one (compound 128);

2-[1-(3,4-dihydroxyphenyl)ethylidene]-6,7-dihydroxy-1-benzofuran-3(2H)-one (compound 129);

2-[1-(2,4-dihydroxyphenyl)ethylidene]-6,7-dihydroxy-1-benzofuran-3(2H)-one (compound 130);

2-[1-(3-fluoro-4-hydroxyphenyl)ethylidene]-6,7-dihydroxy-1-benzofuran-3(2H)-one (compound 131);

2-[1-(3-hydroxy-4-fluorophenyl)ethylidene]-6,7-dihydroxy-1-benzofuran-3(2H)-one (compound 132);

2-benzyl-6,7-dihydroxy-1-benzofuran-3(2H)-one (compound 133);

6,7-dihydroxy-2-(4-hydroxybenzyl)-1-benzofuran-3(2H)-one (compound 134);

6,7-dihydroxy-2-(3-hydroxybenzyl)-1-benzofuran-3(2H)-one (compound 135);

2-(2,4-dihydroxybenzyl)-6,7-dihydroxy-1-benzofuran-3(2H)-one (compound 136);

2-[1-(3,4-dihydroxyphenyl)ethyl]-6,7-dihydroxy-1-benzofuran-3(2H)-one (compound 137);

6,7-dihydroxy-2-(3-pyridinylmethyl)-1-benzofuran-3(2H)-one (compound 138);

6,7-dihydroxy-2-[(6-methoxy-2-naphthyl)methyl]-1-benzofuran-3(2H)-one (compound 139);

6,7-dihydroxy-2-[(5-methoxy-1H-indol-2-yl)methyl]-1-benzofuran-3(2H)-one (compound 140);

6,7-dihydroxy-2-[(1-methyl-1H-benzimidazol-2-yl)methyl]-1-benzofuran-3(2H)-one (compound 141);

6,7-dihydroxy-2-[(1-methyl-1H-imidazol-2-yl)methyl]-1-benzofuran-3(2H)-one (compound 142);

6,7-dihydroxy-2-({5-[2-(trifluoromethyl)phenyl]-2-furyl}methyl)-1-benzofuran-3(2H)-one (compound 143); and the pharmaceutically acceptable salt thereof.

A further object of the present invention is to provide a compound of formula (I), as defined above, for use as a medicament, in particular as an anticancer agent. The present invention also provides the use of a compound of formula (I), as defined above, in the preparation of a medicament having anticancer activity.

Another object of the present invention is to provide a method for the preparation of compounds of formula (I). The compounds of formula (I) are obtainable through a synthetic process comprising well known reactions carried out according to conventional techniques.

In addition to the above, it is also clear to the skilled man that most of the compounds of formula (I) of the invention can be advantageously prepared by combining the above described reactions in a combinatorial fashion, for example according to liquid-phase-synthesis techniques, so as to get a combinatorial chemical library of compounds of formula (I).

According to a preferred embodiment of the invention, a compound of formula (I) wherein each of Rₐ and R_{b} represents, independently, hydrogen, C₁-C₆ alkyl or C₁-C₆ alkylcarbonyl or, Rₐ and R_{b}, taken together, represent methylene, and [symbol] Q represents a group of formula (b) or (d), i.e. a compound of formula (IG) wherein -T represents a group of formula

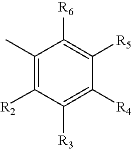

wherein R₂, R₃, R₄, R₅ and R₆ are as defined in formula (I) under (b) or -T represent R₈ as defined in formula (I) under (d), and R₁ is as defined in formula (I) under (b) or (d), can be prepared by a process comprising: reduction by standard methods of the isolated double bond, for example by catalytic hydrogenation with Pd on carbon in organic solvents such as, e.g., methanol, ethanol or DMF, of a compound of formula (I) wherein Rₐ and R_{b} are as defined above and [symbol] Q represents a group (a) or (c), i.e. a compound of formula (IF) wherein -T is a group of formula

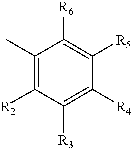

wherein R₂, R₃, R₄, R₅ and R₆ are as defined in formula (I) under (a) or -T is a group R₇ as defined in formula (I) under (c), and R₁ is as defined in formula (I) under (a) or (c), as shown in Scheme 1 below.

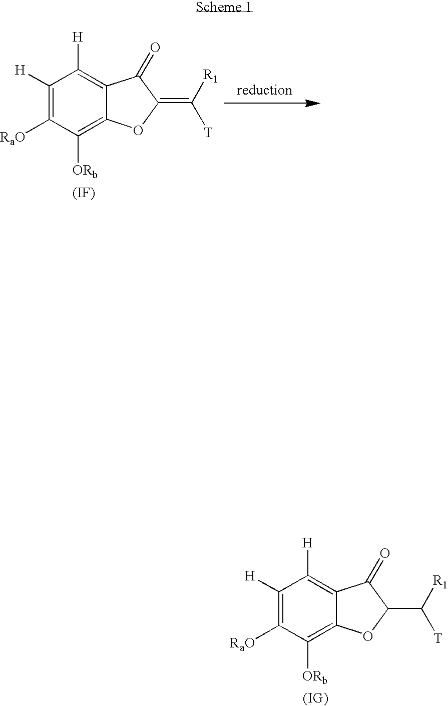

A further object of the present invention is to provide a method for the preparation of compounds of formula (I) wherein Rₐ and R_{b} are as defined above and [symbol] Q is a group of formula (a), (c) or (e).

According to a preferred embodiment of the invention a compound of formula (I) wherein Rₐ and R_{b} are as defined above and [symbol] Q is a group of formula (a), (c) or (e), i.e. a compound of formula (IH) wherein Rₐ and R_{b} are as defined above and wherein -U is a group of formula

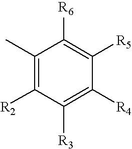

wherein R₂, R₃, R₄, R₅ and R₆ are as defined in formula (I) under (a), or -U is R₇ as defined in formula (I) under (c), or -U is a group of formula

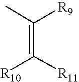

wherein R₉, R₁₀ and R₁₁ are as defined under (e) above, and R₁ is as defined in formula (I) under (a), (c) or (e), can be prepared by standard procedures as described in the literature, typically by a process comprising condensation in acidic medium (J.O.C.1955, 77, 4622; J.Prakt.Chem. 1998, 340, 271; J.C.S. Perkin Trans.1, 1972, 2128; JACS 1942, 64, 382) or in neutral medium (Tetr.Lett. 1992, 33, 5937) of 3(2H)-benzofuranones of formula (II) wherein Rₐ and R_{b} are as defined above, with aldehydes or ketones of general formula (III), as reported in Scheme 2.

Typically this condensation reaction is carried out at temperatures ranging from room temperature to 100° C. using glacial acetic acid as the solvent in the presence of concentrated hydrochloric acid or using a solvent such as ethanol, methanol, dichloromethane, ethyl acetate, THF, DMF in the presence of acetic acid and a base, e.g. an organic base as piperidine, piperazine, morpholine, dialkylamines and the like, for a period of time from 1 to 72 hours. Alternatively the reaction can be made in an organic solvent such as dichloromethane, ethyl acetate, methanol, ethanol, THF, DMF and their mixtures using neutral alumina as the condensing agent, usually at temperatures ranging from room to reflux temperature for a period of time from 1 to 72 hours.

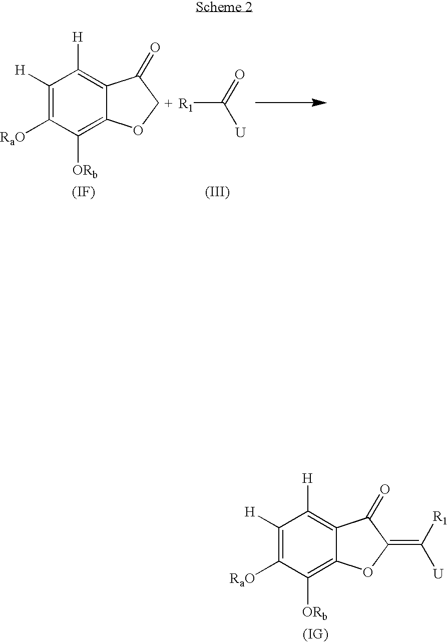

where Rₐ, R_{b}, R₁ and R are as described above.

The above process is an analogy process, which can be carried out according to well-known methods.

Likewise, the salification of a compound of formula (I) or the conversion of its salt into the free compound (I), carried out according to well-known procedures in the art, are still within the scope of the invention.

As it will be really appreciated by the man skilled in the art, when preparing the compounds of formula (I) according to an object of the invention, optional functional groups within both the starting materials or the intermediates thereof which could give rise to unwanted side reactions, need to be properly protected according to conventional techniques. Likewise, the conversion of these latter into the free deprotected compounds can be carried out according to known procedures.

In addition to the above, it is also clear to the skilled man that most of the compounds of formula (I), i.e. the compounds of formula (I) wherein Rₐ and R_{b} are hydrogen and [symbol] Q is a group (a), (c) or (e) in which R₁ is hydrogen and R₂-R₇, R₉-R₁₁ are as defined in formula (I) above, can be advantageously prepared by combining the above described reactions in a combinatorial fashion, for example according to liquid-phase-synthesis (LPS) techniques, so as to get a combinatorial chemical library of compounds of formula (I).

It is therefore a further object of the invention a library of two or more compounds of formula (I) or a pharmaceutically acceptable salt thereof,

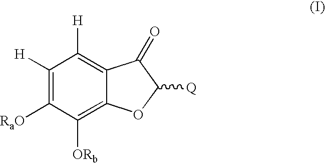

wherein

Rₐ and R_{b} represent hydrogen;

[symbol] Q represents a group of formula (a), (c) or (e)

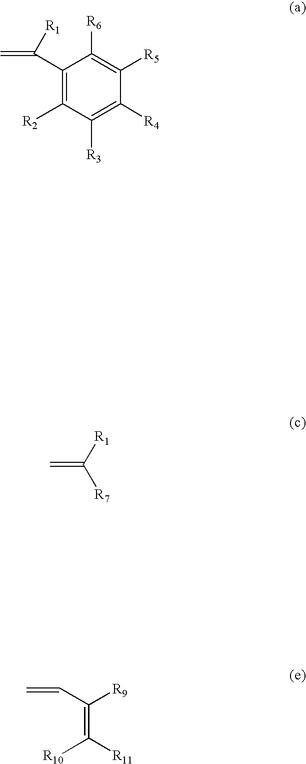

wherein

in a group of formula (a)

R₁ represents hydrogen;

each of R₂, R₅ and R₆ represents, independently, hydrogen, halogen, hydroxy, C₁-C₆ alkyl, haloalkyl, C₁-C₆ alkenyl, C₁-C₆ alkoxy, C₁-C₆ alkenyloxy, aryloxy, arylalkoxy, haloalkoxy, C₁-C₆ alkoxycarbonyl, carboxyl, nitro or cyano; and

each of R₃ and R₄ represents, independently, hydrogen, halogen, hydroxy, C₁-C₆ alkyl, haloalkyl, optionally substituted alkenyl, optionally substituted arylalkenyl, arylalkinyl, aryl, C₁-C₆ alkoxy, aryloxy, arylalkoxy, haloalkoxy, aminoalkoxy, carbalkoxy, C₁-C₆ alkoxycarbonylalkoxy, carboxyl, C₁-C₆ alkoxycarbonyl, acyloxy, amino, dialkylamino, optionally substituted dialkylamino, acylamino, thioalkyl, arylsulfonyl, alkylsulfonyl, arylsulfenyl, alkylsulfenyl, arylsulfanyl, alkylsulfanyl, nitro or cyano;

in a group of formula (c)

R₁ represents hydrogen; and

R₇ represents a fused polycyclic optionally substituted aryl or a monocyclic, bicyclic or tricyclic optionally substituted heteroaryl; and

in a group of formula (e)

R₉ represents hydrogen, C₁-C₆ alkyl, halogen or optionally substituted aryl;

R₁₀ represents C₁-C₆ alkyl, C₁-C₄ alkoxy, carboxyl, alkoxycarbonyl, optionally substituted aryl or optionally substituted heteroaryl and R₁₁ represents hydrogen, halogen or optionally substituted aryl.

All of the compounds of formula (I) which are prepared according to combinatorial chemistry techniques, for instance as reported in Example 2, are herewith conveniently indicated and defined as “products by process”, that is as compounds of formula (I) which are obtainable through a given process.

As such, it is a further object of the present invention a compound of formula (I) which is obtainable, for instance through a combinatorial chemistry technique, by reacting 6,7-dihydroxy-3(2H)-benzofuranone, i.e. a compound of formula (II) wherein Rₐ and R_{b} are both hydrogen, with any one of the aldehydes of formula (III), as set forth in Table I. As an example, these reactions can be carried out in a multireaction apparatus, such as, for example, the Robbins FlexChem™ 96-well reaction blocks, so obtaining the library from compound 8 to compound 88 and from compound 90 to compound 125.

TABLE I
Aldehydes of formula (III)

| | |
|---|---|
| 1. | 2,5-DIHYDROXY BENZALDEHYDE |
| 2. | 3-FLUORO, 2-HYDROXY BENZALDEHYDE |
| 3. | 2,3-METHYLENEDIOXY BENZALDEHYDE |
| 4. | 2,4,6-TRIFLUORO BENZALDEHYDE |
| 5. | 2-HYDROXY, 3-METHOXY BENZALDEHYDE |
| 6. | 3,5-DIMETHYL BENZALDEHYDE |
| 7. | 3,4,5-TRIHYDROXY BENZALDEHYDE |
| 8. | 4-CHLORO, 3-FLUORO BENZALDEHYDE |
| 9. | 4-(BENZYLOXY) BENZALDEHYDE |
| 10. | 4-HYDROXY, 3-CARBOXY BENZALDEHYDE |
| 11. | 5-BROMO, 2-HYDROXY BENZALDEHYDE |
| 12. | 3-CARBOXY BENZALDEHYDE |
| 13. | 4-(PHENYLETHYNYL) BENZALDEHYDE |
| 14. | 3,5-DITERT-BUTYL, 2-HYDROXY BENZALDEHYDE |
| 15. | 3,5-DIHYDROXY BENZALDEHYDE |
| 16. | 4-FORMYLPHENYL-2-PROPENOIC ACID |
| 17. | 3,4-DIHYDROXY, 5-METHOXY BENZALDEHYDE |
| 18. | 2-FLUORO, 4-(TRIFLUOROMETHYL) BENZALDEHYDE |
| 19. | 3,4-DIMETHYL BENZALDEHYDE |
| 20. | 3-FLUORO-4-(TRIFLUOROMETHYL) BENZALDEHYDE |
| 21. | 3-BROMO, 5-CHLORO, 2-HYDROXY BENZALDEHYDE |
| 22. | 4-(DIMETHYLAMINO), 2-METHOXY BENZALDEHYDE |
| 23. | 4-(BENZYLOXY), 2-HYDROXY BENZALDEHYDE |
| 24. | 4-(BENZYLOXY), 2-METHOXY BENZALDEHYDE |
| 25. | 2-FLUORO, 4-CHLORO BENZALDEHYDE |
| 26. | 2-(DIFLUOROMETHOXY) BENZALDEHYDE |
| 27. | 2-VINYL BENZALDEHYDE |
| 28. | 2,4-DIMETHOXY, 6-METHOXYCARBONYL |
| | BENZALDEHYDE |
| 29. | 2-CYANO BENZALDEHYDE |
| 30. | 2,3-DICHLORO BENZALDEHYDE |
| 31. | 4-(DIETHYLAMINO) BENZALDEHYDE |
| 32. | 2,4-DIMETHOXY, 3-METHYL BENZALDEHYDE |
| 33. | 2,3,4,5,6 PENTAMETHYL BENZALDEHYDE |
| 34. | 2-BROMO, 4,5-DIMETHOXY BENZALDEHYDE |
| 35. | 3,5-DIMETHOXY BENZALDEHYDE |
| 36. | 3,5-DIMETHOXY, 4-(ACETOXY) BENZALDEHYDE |
| 37. | 3-ETHOXY, 4-METHOXY BENZALDEHYDE |
| 38. | 2,4-DIFLUORO BENZALDEHYDE |
| 39. | 2,5-DIFLUORO BENZALDEHYDE |
| 40. | 2,6-DIFLUORO BENZALDEHYDE |
| 41. | 4-BUTOXY BENZALDEHYDE |
| 42. | 3-CHLORO, 4-FLUORO BENZALDEHYDE |
| 43. | 2,3,6-TRICHLORO BENZALDEHYDE |
| 44. | 3,5-DIFLUORO BENZALDEHYDE |
| 45. | 2,3-DIFLUORO BENZALDEHYDE |
| 46. | 2,3,5-TRICHLORO BENZALDEHYDE |
| 47. | 5-BROMO, 2,4-DIMETHOXY BENZALDEHYDE |
| 48. | 2,6-DIMETHOXY BENZALDEHYDE |
| 49. | 4-HEXYLOXY BENZALDEHYDE |
| 50. | 3-METHYL, 4-METHOXY BENZALDEHYDE |
| 51. | 4-(ACETOXY) BENZALDEHYDE |
| 52. | 4-PROPOXY BENZALDEHYDE |
| 53. | 2,3-METHYLENEDIOXY BENZALDEHYDE |
| 54. | 4-PHENOXY BENZALDEHYDE |
| 55. | 4-(BENZYLOXY), 3-METHOXY BENZALDEHYDE |
| 56. | 2-CHLORO, 6-FLUORO BENZALDEHYDE |
| 57. | 2,3-DIMETHYL, 4-METHOXY BENZALDEHYDE |
| 58. | 2,5-DIMETHYL, 4-METHOXY BENZALDEHYDE |
| 59. | 2,3,4,5,6 PENTAFLUORO BENZALDEHYDE |
| 60. | 3-PHENOXY BENZALDEHYDE |
| 61. | 3-(4-CHLOROPHENOXY) BENZALDEHYDE |
| 62. | 3-(4-METHOXYPHENOXY) BENZALDEHYDE |
| 63. | 3-(4-METHYLPHENOXY) BENZALDEHYDE |
| 64. | 4-(3-DIMETHYLAMINO) PROPOXY BENZALDEHYDE |
| 65. | 2-FLUORO, 4-BROMO BENZALDEHYDE |
| 66. | 2,4-DIETHOXY, 3-METHYL BENZALDEHYDE |
| 67. | 2-CHLORO, 5-(TRIFLUOROMETHYL) BENZALDEHYDE |
| 68. | 4-FLUORO, 2-(TRIFLUOROMETHYL) BENZALDEHYDE |
| 69. | 2-FLUORO, 6-(TRIFLUOROMETHYL) BENZALDEHYDE |
| 70. | 4-TERT-BUTYL BENZALDEHYDE |
| 71. | 2,3,5,6-TETRAFLUORO BENZALDEHYDE |
| 72. | 4-(TRIFLUOROMETHOXY) BENZALDEHYDE |
| 73. | 4-(DIBUTYLAMINO) BENZALDEHYDE |
| 74. | 4-[BIS(2-CYANOETHYL)AMINO BENZALDEHYDE |
| 75. | 3-(TRIFLUOROMETHOXY) BENZALDEHYDE |
| 76. | 2-CHLORO, 4-FLUORO BENZALDEHYDE |
| 77. | 2-METHYL, 3-FLUORO BENZALDEHYDE |
| 78. | 2-FLUORO, 3-(TRIFLUOROMETHYL) BENZALDEHYDE |
| 79. | 4-(DIFLUOROMETHOXY) BENZALDEHYDE |
| 80. | 2,5-BIS(TRIFLUOROMETHYL) BENZALDEHYDE |
| 81. | 4-FLUORO, 3-(TRIFLUOROMETHYL) BENZALDEHYDE |
| 82. | 3-PYRIDINECARBOXALDEHYDE |
| 83. | 6-HYDROXYCHROMENE-3-CARBOXALDEHYDE |
| 84. | 6-METHOXY-2-NAPHTHALDEHYDE |
| 85. | 5-METHYL-2-THIOPHENECARBOXALDEHYDE |
| 86. | 5-METHOXYINDOLE-3-CARBOXALDEHYDE |
| 87. | 1-METHYL-2-FORMYLBENZIMIDAZOLE |
| 88. | 4-HYDROXY-3-METHOXYCINNAMALDEHYDE |
| 89. | 3,3-DIPHENYL ACROLEIN |
| 90. | ALPHA-METHYLCINNAMALDEHYDE |
| 91. | 4-DIMETHYLAMINOCINNAMALDEHYDE |
| 92. | 1-ACETYL-3-INDOLECARBOXALDEHYDE |
| 93. | 5-METHYLIMIDAZOLE-4-CARBOXALDEHYDE |
| 94. | 5-FORMYLURACIL |
| 95. | 1-METHYL-2-IMIDAZOLECARBOXALDEHYDE |
| 96. | 7-FORMYLINDOLE |
| 97. | 3-METHYLBENZO[B]THIOPHENE-2-CARBOXALDEHYDE |
| 98. | 1,4-BENZODIOXAN-6-CARBOXALDEHYDE |
| 99. | 9-ANTHRALDEHYDE |
| 100. | 1-PYRENECARBOXALDEHYDE |
| 101. | 5-ACETOXYMETHYL-2-FURALDEHYDE |
| 102. | PHENANTHRENE-9-CARBOXALDEHYDE |
| 103. | 2-FLUORENECARBOXALDEHYDE |
| 104. | 10-CHLORO-9-ANTHRALDEHYDE |
| 105. | 10-METHYLANTHRACENE-9-CARBOXALDEHYDE |
| 106. | 5-[2-(TRIFLUOROMETHYL)PHENYL]FURFURAL |
| 107. | 5-(2-CHLOROPHENYL)FURFURAL |
| 108. | 4,5-DIMETHYL-2-FURANCARBOXALDEHYDE |
| 109. | 5-BROMO-2-FURALDEHYDE |
| 110. | 5-(3-CHLOROPHENYL)-2-FURALDEHYDE |
| 111. | 1-(PHENYLSULFONYL)-2-PYRROLECARBOXALDEHYDE |
| 112. | 5-(3-TRIFLUOROMETHYLPHENYL)FURAN-2- |
| | CARBOXALDEHYDE |
| 113. | 5-ETHYL-2-FURALDEHYDE |
| 114. | 5-CHLORO-2-THIOPHENECARBOXALDEHYDE |
| 115. | 5-CHLORO-3-METHYL-1-PHENYL-1H-PYRAZOLE-4- |
| | CARBALDEHYDE |
| 116. | 5-FORMYL-2,4-DIMETHOXY-PYRIMIDINE |
| 117. | 3-(4-TERT-BUTYL-PHENYL)-2-METHYL-PROPENAL |

Non-commercially available benzofuranones (II) were prepared by standard methods as described in the literature, for example, in Synth. Comm. 1995, 25, 915, ibid. 1994, 24, 29, J.O.C. 1988, 53, 423, J.A.C.S. 1956, 78, 1505. Generally, aldehydes and ketones (III) are commercially available or preparable with synthetic methods as described in the literature [e.g. Tetrahedron 1996, 52, 3889, WO0015239 (2000), WO0015240 (2000), WO9418200 (1994), EP61907 (1982)].

When in compound (I) free hydroxyl groups are present, a step of deprotection of a C₁-C₆ alkoxy (typically methoxy, ethoxy or benzyloxy) substituted precursor, or by deprotection of a silyloxy (typically trimethylsilyloxy, triisopropylsilyloxy, triethylsilyloxy, t-butyldimethylsilyloxy, or phenyldimethylsilyloxy) substituted precursor may be desirable. For example typical demethylation procedures require the use of an aqueous acid, like 57% HI, 48% HBr, eventually in the presence of glacial acetic acid, at temperatures ranging from room to refluxing temperature for reaction time ranging from 1 to 72 hrs. In other cases the use of a Lewis acid, like BBr₃, BCl₃, AlCl₃ and similar reagents, is preferred, in the presence of a suitable organic solvent like methylene chloride, benzene, toluene, and the like, at temperatures ranging from −78° to 150° C. for 1 to 72 hrs. Most preferred among the previous Lewis acids is BBr₃.

All of the compounds of formula (I) which are-prepared according to combinatorial chemistry techniques, for instance as reported in the examples, whenever appropriate in the form of pharmaceutically acceptable salts, are herewith conveniently indicated and defined as “products by process”, that is as compounds of formula (I) which are obtainable through a given process.

The compounds of formula (I), (IA), (IB), (IC), (ID) and (IE) are herein defined as the “compounds of the present invention”, the “compounds of the invention” and/or the “active principles of the pharmaceutical compositions of the invention”.

The compounds of the invention can be administered in a variety of dosage forms, e.g. orally, in the form of tablets, capsules, lozengers, liquid solutions or suspensions; rectally, in the form of suppositories; parenterally, e.g. intramuscularly, intravenously, intradermally or subcutaneously; or topically. The dosage depends upon, for example, the compound of the invention employed, the age, weight, condition of the patient and administration route; specific dosage regimens can be fit to any particular subject on the basis of the individual need and the professional judgement of the person administering or supervising the administration of the aforesaid compounds. For example, the dosage adopted for the administration to adult humans can range from 0.001 to 100 mg of compound of the invention per kg of body weight; a particularly preferred range can be from 0.1 to 10 mg of compound of the invention per kg of body weight. The dosages can be administered at once or can be divided into a number of smaller doses to be administered at varying intervals of time.

As already mentioned above, pharmaceutical compositions containing, as an active ingredient, a compound of the present invention or a pharmaceutically acceptable salt thereof in association with a pharmaceutically acceptable excipient, are also within the scope of the present invention. These pharmaceutical compositions contain an amount of active ingredient, which is therapeutically effective to display, for example, antileukemic and/or antitumor activity. There can also be included as a part of the pharmaceutical compositions according to the invention, pharmaceutically acceptable binding agents and/or adjuvant materials. The active ingredients can also be mixed with other active principles, which do not impair the desired action and/or supplement the desired action.

The pharmaceutical compositions containing the compounds of the invention are usually prepared following conventional methods and can be administered in a pharmaceutically suitable form. For example, the solid oral forms can contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents, e.g. starches, arabic gums, gelatin, methylcellulose, microcrystalline cellulose, carboxymethylcellulose or polyvinyl pyrrolidone; diaggregating agents, e.g. a starch, alginic acid, alginates or sodium starch glycolate; effervescing mixtures; dyestuffs; sweetening agents, e.g. sucrose or saccharin; flavouring agents, e.g. peppermint, methylsalicylate or orange flavouring; wetting agents, such as lecithin, polysorbates, laurylsulphates; and, in general, non-toxic and pharmacologically inactive substances used in pharmaceutical formulations. When the dosage unit form is a capsule, it can contain, in addition to material of the above type, a liquid carrier such as, e.g., a fatty oil.

Said pharmaceutical preparations can be manufactured in known manner, for example, by means of mixing, granulating, tabletting, sugar-coating or film-coating processes. The liquid dispersions for oral administration can be, e.g. syrups, emulsions and suspensions.

The syrups can contain as carrier, for example, saccharose or saccharose with glycerine and/or mannitol and/or sorbitol; in particular, a syrup to be administered to diabetic patients can contain as carriers only products not metabolizable to glucose, or metabolizable in very small amount to glucose, for example sorbitol.

The suspensions and the emulsions can contain as carrier, for example, a natural gum, agar, sodium alginate, pectin, methylcellulose, carboxymethylcellulose, or polyvinyl alcohol. The suspensions or solutions for intramuscular injections can contain, together with the active compound, a pharmaceutically acceptable carrier, e.g. sterile water, olive oil, ethyl oleate, glycols, e.g. propylene glycol, and, if desired, a suitable amount of lidocaine hydrochloride. The solutions for intravenous injections or infusions can contain as carrier, for example, sterile water, or preferably they can be in the form of sterile, aqueous, isotonic saline solution.

The solutions or suspensions for parenteral therapeutic administration can also contain antibacterial agents, such as benzyl alcohol or methyl parabens; antioxidants, such as ascorbic acid or sodium bisulphite; chelating agents, such as ethylenediaminetetraacetic acid; buffers, such as acetates, citrates or phosphates and agents for the adjustment of tonicity, such as sodium chloride or dextrose. The parenteral preparation can be enclosed in ampoules, disposable syringes or multiple dose vials made of glass or plastic.

The suppositories can contain together with the active compound a pharmaceutically acceptable carrier, e.g., coca-butter, polyethylene glycol, a polyoxyethylene sorbitan fatty acid ester surfactant or lecithin.

Compositions for topical application, such as, e.g., creams, lotions or pastes, can be, e.g., prepared by admixing the active ingredient with a conventional oleaginous or emulsifying excipient.

### Biological Activity

The compounds of formula (I), are active as telomerase inhibitors as they gave positive results when tested according to the following procedures.

The compounds of formula (I) are therefore useful to manage the unregulated proliferation of tumor cells, hence in therapy in the treatment of various tumors such as, for instance, carcinomas, e.g. mammary carcinoma, lung carcinoma, bladder carcinoma, colon carcinoma, ovary and endometrial tumors, sarcomas, e.g. soft tissue and bone sarcomas, and the hematological malignancies such as, e.g., leukemias.

The telomerase activity of the compounds has been evaluated using a Flash Plate-based assay. The method proved to be sensitive, accurate and able to reproducibly identify compounds that inhibit telomerase activity in a dose-dependent manner.

The assay mixture is constituted of:

telomerase enzyme diluted in a buffer, the composition of which has been selected to maintain the enzyme activity stable along the duration of the assay.

dNTPs, deoxynucleotides 5′-triphosphate.

biotinylated oligo as primer.

increasing concentrations of test compounds/positive control.

After two hours of incubation at 37° degrees the telomeric repeats added by telomerase are evaluated by hybridization in solution with a 3′-radioactive labeled short oligonucleotide probe. The extent of hybridization is then quantitated by transferring the reaction mixture in a streptavidin-coated flash plate, where the binding between biotin and streptavidin occurs. The telomerase activity is proportional to the radioactivity measured and the inhibitory activity of the compounds is evaluated as IC₅₀ using the Sigma Plot fit program.

The IC₅₀ values for the compounds of the present invention were determined according to the above-described method. Results of the IC₅₀ values' determinations for a representative selection of compounds of the invention are shown in Table 2.

TABLE 2

| Compound | IC₅₀ (μM) |
|---|---|
| 1 | 0.3 |
| 2 | 13 |
| 3 | 11 |
| 4 | 1.5 |
| 5 | 8 |
| 89 | 7 |

According the data reported in Table 2 the compounds of the present invention possess a telomerase inhibitory activity. A human or animal body can thus be treated by a method, which comprises the administration thereto of a pharmaceutically effective amount of a compound of formula (I) or a salt thereof. The condition of the human or animal can thereby be improved.

The compounds of the invention can be administered either as single agents or, alternatively, in combination with one or more anti-cancer agent including, for example, topoisomerase inhibitors, antimetabolites, alkylating agents, antibiotics, antimicrotubule agents, hormonal agents, immunological agents, interferon-type agents, cyclooxygenase inhibitors (e.g. COX-2 inhibitors), metallomatrixprotease inhibitors, kinase inhibitors, tyrosine kinase inhibitors, anti-growth factor receptor agents, anti-HER agents, anti-EGFR agents, farnesyl transferase inhibitors, ras-raf signal transduction pathway inhibitors, cell cycle inhibitors, tubulin binding agents and anti-angiogenesis agents. Combinations of drugs are administered in an attempt to obtain a synergistic effect on most cancers, e.g., carcinomas, melanomas, sarcomas, lymphomas and leukemias and/or to reduce or eliminate emergence of drug-resistant cells and/or to reduce side effects to each drug.

Therefore a further aspect of the present invention is a combined anti-cancer therapy which comprises administering a compound according to the invention with at least one other anti-cancer agent. The combined or coordinated use of active substances provides improved therapeutic effect over employing the single agents alone. Compounds of formula (I) can be combined with at least one other anti-cancer agent in a fixed pharmaceutical formulation or can be administered with at least one other anti-cancer agent in any desired order.

Therefore a further object of the invention is a product or kit comprising a compound of formula (I) of the invention and one or more anti-cancer agents for coordinated (i.e., simultaneous, separate and/or sequential) use in anticancer therapy. Anti-cancer agents suitable for combination with the compounds of the present invention include, but are not limited to:

topoisomerase I inhibitors comprising, for example, epipodophyllotoxins such as, e.g. etoposide and teniposide; camptothecin and camptothecin derivatives including, e.g., iririotecan, SN-38, topotecan, 9-amino-camptothecin, 10,11-Methylenedioxy camptothecin: and 9-nitro-camptothecin (rubitecan);

alkylating agents including nitrogen mustards such as, e.g., mechlorethamine, chlorambucil, melphalan, uracil mustard and estramustine; alkylsulfonates such as, e.g., busulfan improsulfan and piposulfan; oxazaphosphorines such as e.g., ifosfamide, cyclophosphamide, perfosfamide, and trophosphamide; platinum derivatives such as, e.g., oxaliplatin, carboplatin and cisplatin; nitrosoureas such as, e.g., carmustine, lomustine and streptozocin;

antimitotic agents including taxanes such as, e.g., paclitaxel and docetaxel; vinca alkaloids such as, e.g., vincristine, vinblastine, vinorelbine and vindesine; and novel microtubule agents such as, e.g., epothilone analogs, discodermolide analogs and eleutherobin analogs;

antimetabolites including purines such as, e.g., 6-mercaptopurine, thioguanine, azathioprine, allopurinol, cladribine, fludarabine, pentostatin, and 2-chloro adenosine; fluoropyrimidines such as, e.g., 5-FU, fluorodeoxyuridine, ftorafur, 5′-deoxyfluorouridine, UFT, S-1 and capecitabine; and pyrimidine nucleosides such as, e.g., deoxycytidine, cytosine arabinoside, 5-azacytosine, gemcitabine, and 5-azacytosine-arabinoside; antimetabolites (for example antifolates like methotrexate, fluoropyrimidines like 5-fluorouracil, purine and adenosine analogues, cytosine arabinoside;

hormones, hormonal analogues and hormonal antagonists including antiestrogens (for example tamoxifen, toremifen, raloxifene, droloxifene and iodoxyfene), progestogens (for example megestrol and acetate), aromatase inhibitors (for example anastrozole, letrazole, borazole and exemestane), antiprogestogens, antiandrogens (for example flutamide, nilutamide, bicalutamide and cyproterone acetate), LHRH agonists and antagonists (for example gosereline acetate and luprolide) and inhibitors of testosterone 5a-dihydroreductase (for example finasteride;

antitumor antibiotics including anthracyclines and anthracenediones such as, e.g., doxorubicin, daunorubicin, epirubicin, idarubicin and mitoxantrone;

farnesyltransferase inhibitors including, for example, SCH 44342, RPR 113228, BZA 5B and PD 161956;

anti-invasion agents (for example metalloproteinase inhibitors such as, e.g., marimastat and inhibitors of urokinase plasminogen activator receptor functions);

inhibitors of growth factor (for example, EGF, FGF, platelet derived growth factor and hepatocyte growth factor) functions including growth factor antibodies, growth factor receptor antibodies, tyrosine kinase inhibitors and serine/threonine kinase inhibitors;

antiangiogenic agents such as, for example, linomide, inhibitors of integrin avβ3 function, angiostatin, razoxin, SU 5416, SU 6668, AGM 1470 (TNP-470), a synthetic analogue of fumagillin a naturally secreted product of the fungus Aspergillus fumigates fresenius, platelet factor 4 (endostatin), thalidomide, marimastat (BB-2516) and batimastat (BB-94);

cyclooxygenase (COX) inhibitors, preferably COX-2 inhibitors such as, for example, celecoxib, parecoxib, rofecoxib, valecoxib and JTE 5222; and

cell cycle inhibitors such as, e.g., flavopyridols.

In a further aspect of this invention, a method is provided for treating a cancer comprising administering to a patient in need of such treatment a therapeutically effective amount of a substituted aurones as defined in formula (I) above or a pharmaceutically acceptable salt thereof and a therapeutically effective amount of at least another anti-cancer agent.

The following examples illustrate but do not limit the invention:

### EXAMPLE 1

### 2-(3,4-dichlorobenzylidene)-6,7-dihydroxy-1-benzofuran-3(2H)-one (compound 5)

To a solution of 6,7-dihydroxy-3(2H)-benzofuranone (100 mg, 0.6 mmol) and 3,4-dichloro benzaldehyde (105 mg, 0.6 mmol) in glacial acetic acid (5 mL) 37% HCl (0.2 mL) is added and the solution stirred for 3 hours at room temperature. After removal of acetic acid under reduced pressure the reaction mixture is diluted with water and the precipitate filtered. The solid is washed thoroughly with water and diethyl ether and dried. Yield: 85%. ¹H-NMR (400 Mhz, DMSOd₆), ppm: 6.74 (1H, d, J=8.3 Hz), 6.78 (1H, s), 7.15 (1H, d, J=8.3 Hz), 7.74 (1H, d, J=8.2 Hz), 8.03 (1H, dd, J=8.2, 2 Hz), 8.24 (1H, d, J=2 Hz); MS m/z 324 [M+H]⁺.

By analogous procedure the following compound was prepared:

2-(3,4-dihydroxybenzylidene)-6,7-dimethoxy-1-benzofuran-3(2H)-one (compound 6) (yield: 65%). ¹H-NMR (400 Mhz, DMSOd₆), ppm: 3.93 (3H, s), 4.02 (3H, s), 6.7 (1H, s), 6.84 (1H, d, J=8.2 Hz), 7.0 (1H, d, J=8.6 Hz), 7.24 (1H, dd, J=8.2, 2 Hz), 7.43 (1H, d, J=2 Hz), 7.47 (1H, d, J=8.6 Hz), 9.32 (1H, s), 9.7 (1H, s); MS m/z 315 [M+H]⁺.

### EXAMPLE 2

### 2-(3,5-dihydroxybenzylidene)-6,7-dihydroxy-1-benzofuran-3(2H)-one (compound 22)

To a solution of 3,4-dihydroxy-3(2H)-benzofuranone (11.2 mg, 0.068 mmol) in anhydrous EtOH (1 mL) it was added, under stirring, glacial acetic acid (15.5 mcL, 0.27 mmol) followed by piperidine (24 mcL, 0.23 mmol). To this mixture 3,5-dihydroxy benzaldehyde (6.2 mg, 0.045 mmol) was added and the reaction mixture was heated for 18 h at 60° C. The solvent was evaporated and the crude product was purified by preparative HPLC-MS. The product was obtained as a yellow solid (5.8 mg, 0.02 mmol, 44%).

By analogous procedure and using the Robbins FlexChem™ 96 well-reaction blocks, the following compounds were prepared:

Group 1

2-(2,5-dihydroxybenzylidene)-6,7-dihydroxy-1-benzofuran-3(2H)-one (compound 8);

2-(3-fluoro-2-hydroxybenzylidene)-6,7-dihydroxy-1-benzofuran-3(2H)-one (compound 9);

6,7-dihydroxy-2-[(7-methoxy-1,3-benzodioxol-5-yl)methylene]-1-benzofuran-3(2H)-one (compound 10);

6,7-dihydroxy-2-(2,4,6-trifluorobenzylidene)-1-benzofuran-3(2H)-one (compound 11);

6,7-dihydroxy-2-(2-hydroxy-3-methoxybenzylidene)-1-benzofuran-3(2H)-one (compound 12);

2-(3,5-dimethylbenzylidene)-6,7-dihydroxy-1-benzofuran-3(2H)-one (compound 13);

2-(3,4,5-trihydroxybenzylidene)-6,7-dihydroxy-1-benzofuran-3(2H)-one (compound 14);

2-(4-chloro-3-fluorobenzylidene)-6,7-dihydroxy-1-benzofuran-3(2H)-one (compound 15);

2-[4-(benzyloxy)benzylidene]-6,7-dihydroxy-1-benzofuran-3(2H)-one (compound 16);

5-[(6,7-dihydroxy-3-oxo-1-benzofuran-2(3H)-ylidene)methyl]-2-hydroxybenzoic acid (compound 17);

2-(5-bromo-2-hydroxybenzylidene)-6,7-dihydroxy-1-benzofuran-3(2H)-one (compound 18);

3-[(6,7-dihydroxy-3-oxo-1-benzofuran-2(3H)-ylidene)methyl]benzoic acid (compound 19);

6,7-dihydroxy-2-[4-(phenylethynyl)benzylidene-1-benzofuran-3(2H)-one (compound 20);

2-(3,5-ditert-butyl-2-hydroxybenzylidene)-6,7-dihydroxy-1-benzofuran-3(2H)-one (compound 21);

3-{4-[(6,7-dihydroxy-3-oxo-1-benzofuran-2(3H)-ylidene)methyl]phenyl}-2-propenoic acid (compound 23);

2-(3,4-dihydroxy-5-methoxybenzylidene)-6,7-dihydroxy-1-benzofuran-3(2H)-one (compound 24);

2-[2-fluoro-4-(trifluoromethyl)benzylidene]-6,7-dihydroxy-1-benzofuran-3(2H)-one (compound 25);

6,7-dihydroxy-2-(3,4-dimethylbenzylidene)-1-benzofuran-3(2H)-one (compound 26);

2-[3-fluoro-4-(trifluoromethyl)benzylidene]-6,7-dihydroxy-1-benzofuran-3(2H)-one (compound 27);

2-(3-bromo-5-chloro-2-hydroxybenzylidene)-6,7-dihydroxy-1-benzofuran-3(2H)-one (compound 28);

2-[4-(dimethylamino)-2-methoxybenzylidene]-6,7-dihydroxy-1-benzofuran-3(2H)-one (compound 29);

2-[4-(benzyloxy)-2-hydroxybenzylidene]-6,7-dihydroxy-1-benzofuran-3(2H)-one (compound 30);

2-[4-(benzyloxy)-2-methoxybenzylidene]-6,7-dihydroxy-1-benzofuran-3(2H)-one (compound 31);

2-(2-fluoro-4-chlorobenzylidene)-6,7-dihydroxy-1-benzofuran-3(2H)-one (compound 32);

2-(2-(difluoromethoxy)benzylidene]-6,7-dihydroxy-1-benzofuran-3(2H)-one (compound 33);

6,7-dihydroxy-2-(2-vinylbenzylidene)-1-benzofuran-3(2H)-one (compound 34);

methyl 2-[(6,7-dihydroxy-3-oxo-1-benzofuran-2(3H)-ylidene)methyl]-3,5-dimethoxybenzoate (compound 35);

2-[(6,7-dihydroxy-3-oxo-1-benzofura-2(3H)-ylidene)methyl)benzonitrile (compound 36);

2-(2,3-dichlorobenzylidene)-6,7-dihydroxy-1-benzofuran-3(2H)-one (compound 37);

2-[4-(diethylamino)benzylidene]-6,7-dihydroxy-1-benzofuran-3(2H)-one (compound 38);

2-(2,4-dimethoxy-3-methylbenzylidene)-6,7-dihydroxy-1-benzofuran-3(2H)-one (compound 39);

6,7-dihydroxy-2-(2,3,4,5,6-pentamethylbenzylidene)-1-benzofuran-3(2H)-one (compound 40);

2-(2-bromo-4,5-dimethoxybenzylidene)-6,7-dihydroxy-1-benzofuran-3(2H)-one (compound 41);

2-(3,5-dimethoxybenzylidene)-6,7-dihydroxy-1-benzofuran-3(2H)-one (compound 42);

4-[(6,7-dihydroxy-3-oxo-1-benzofuran-2(3H)-ylidene)methyl]-2,6-dimethoxyphenyl acetate (compound 43);

2-(3-ethoxy-4-methoxybenzylidene)-6,7-dihydroxy-1-benzofuran-3(2H)-one (compound 44);

2-(2,4-difluorobenzylidene)-6,7-dihydroxy-1-benzofuran-3(2H)-one (compound 45);

2-(2,5-difluorobenzylidene)-6,7-dihydroxy-1-benzofuran-3(2H)-one (compound 46);

2-(2,6-difluorobenzylidene)-6,7-dihydroxy-1-benzofuran-3(2H)-one (compound 47);

2-(4-butoxybenzylidene)-6,7-dihydroxy-1-benzofuran-3(2H)-one (compound 48);

2-(3-chloro-4-fluorobenzylidene)-6,7-dihydroxy-1-benzofuran- 3(2H)-one (compound 49);

2-(2,3,6-trichlorobenzylidene)-6,7-dihydroxy-1-benzofuran-3(2H)-one (compound 50);

2-(3,5-difluorobenzylidene)-6,7-dihydroxy-1-benzofuran-3(2H)-one (compound 51);

2-(2,3-difluorobenzylidene)-6,7-dihydroxy-1-benzofuran-3(2H)-one (compound 52);

2-(2,3,5-trichlorobenzylidene)-6,7-dihydroxy-1-benzofuran-3(2H)-one (compound 53);

2-(5-bromo-2,4-dimethoxybenzylidene)-6,7-dihydroxy-1-benzofuran-3(2H)-one (compound 54);

2-(2,6-dimethoxybenzylidene)-6,7-dihydroxy-1-benzofuran-3(2H)-one (compound 55);

2-[4-(hexyloxy)benzylidene]-6,7-dihydroxy-1-benzofuran-3(2H)-one (compound 56);

2-(3-methyl-4-methoxybenzylidene)-6,7-dihydroxy-1-benzofuran-3(2H)-one (compound 57);

4-[(6,7-dihydroxy-3-oxo-1-benzofuran-2(3H)-ylidene)methyl]phenyl acetate (compound 58);

6,7-dihydroxy-2-(4-propoxybenzylidene)-1-benzofuran-3(2H)-one (compound 59);

2-(1,3-benzodioxol-4-ylmethylene)-6,7-dihydroxy-1-benzofuran-3(2H)-one (compound 60);

6,7-dihydroxy-2-(4-phenoxybenzylidene)-1-benzofuran-3(2H)-one (compound 61);

2-4-(benzyloxy)-3-methoxybenzylidene]-6,7-dihydroxy-1-benzofuran-3(2H)-one (compound 62);

2-(2-chloro-6-fluorobenzylidene)-6,7-dihydroxy-1-benzofuran-3(2H)-one (compound 63);

2-(2,3-dimethyl-4-methoxybenzylidene)-6,7-dihydroxy-1-benzofuran-3(2H)-one (compound 64);

2-(2,5-dimethyl-4-methoxybenzylidene)-6,7-dihydroxy-1-benzofuran-3(2H)-one (compound 65);

6,7-dihydroxy-2-(2,3,4,5,6-pentafluorobenzylidene)-1-benzofuran-3(2H)-one (compound 66);

6,7-dihydroxy-2-(3-phenoxybenzylidene)-1-benzofuran-3(2H)-one (compound 67);

2-[3-(4-chlorophenoxy)benzylidene]-6,7-dihydroxy-1-benzofuran-3(2H)-one (compound 68);

6,7-dihydroxy-2-[3-(4-methoxyphenoxy)benzylidene]-1-benzofuran-3(2H)-one (compound 69);

6,7-dihydroxy-2-[3-(4-methylphenoxy)benzylidene]-1-benzofuran-3(2H)-one (compound 70);

2-{4-[3-(dimethylamino)propoxy]benzylidene}-6,7-dihydroxy-1-benzofuran-3(2H)-one (compound 71);

2-(2-fluoro-4-bromobenzylidene)-6,7-dihydroxy-1-benzofuran-3(2H)-one (compound 72);

2-(2,4-diethoxy-3-methylbenzylidene)-6,7-dihydroxy-1-benzofuran-3(2H)-one (compound 73);

2-[2-chloro-5-(trifluoromethyl)benzylidene]-6,7-dihydroxy-1-benzofuran-3(2H)-one (compound 74);

2-[4-fluoro-2-(trifluoromethyl)benzylidene]-6,7-dihydroxy-1-benzofuran-3(2H)-one (compound 75);

2-[2-fluoro-6-(trifluoromethyl)benzylidene]-6,7-dihydroxy-1-benzofuran-3(2H)-one (compound 76);

2-(4-tert-butylbenzylidene)-6,7-dihydroxy-1-benzofuran-3(2H)-one (compound 77);

6,7-dihydroxy-2-(2,3,5,6-tetrafluorobenzylidene)-1-benzofuran-3(2H)-one (compound 78);

6,7-dihydroxy-2-[4-(trifluoromethoxy)benzylidene]-1-benzofuran-3(2H)-one (compound 79);

2-[4-(dibutylamino)benzylidene]-6,7-dihydroxy-1-benzofuran-3(2H)-one (compound 80);

2-{4-[bis(2-cyanoethyl)amino]benzylidene}-6,7-dihydroxy-1-benzofuran-3(2H)-one (compound 81);

6,7-dihydroxy-2-[3-(trifluoromethoxy)benzylidene]-1-benzofuran-3(2H)-one (compound 82);

2-(2-chloro-4-fluorobenzylidene)-6,7-dihydroxy-1-benzofuran-3(2H)-one (compound 83);

2-(2-methyl-3-fluorobenzylidene)-6,7-dihydroxy-1-benzofuran-3(2H)-one (compound 84);

2-[2-fluoro-3-(trifluoromethyl)benzylidene]-6,7-dihydroxy-1-benzofuran-3(2H)-one (compound 85);

2-[4-(difluoromethoxy)benzylidene]-6,7-dihydroxy-1-benzofuran-3(2H)-one (compound 86);

2-[2,5-bis(trifluoromethyl)benzylidene]-6,7-dihydroxy-1-benzofuran-3(2H)-one (compound 87);

2-[4-fluoro-3-(trifluoromethyl)benzylidene]-6,7-dihydroxy-1-benzofuran-3(2H)-one (compound 88);

Group 2

6,7-dihydroxy-2-(3-pyridinylmethylene)-1-benzofuran-3(2H)-one (compound 90);

6,7-dihydroxy-2-(6-hydroxy-4H-chromen-3-yl)methylene-1-benzofuran-3(2H)-one (compound 91);

6,7-dihydroxy-2-[(6-methoxy-2-naphthyl)methylene]-1-benzofuran-3(2H)-one (compound 92);

6,7-dihydroxy-2-[(5-methyl-2-thienyl)methylene]-1-benzofuran-3(2H)-one (compound 93);

6,7-dihydroxy-2-[(5-methoxy-1H-indol-2-yl)methylene-1-benzofuran-3(2H)-one (compound 94);

6,7-dihydroxy-2-[(1-methyl-1H-benzimidazol-2-yl)methylene]-1-benzofuran-3(2H)-one (compound 95);

2-[(1-acetyl-1H-indol-3-yl)methylene]-6,7-dihydroxy-1-benzofuran-3(2H)-one (compound 96);

6,7-dihydroxy-2-[(4-methyl-1H-imidazol-5-yl)methylene]-1-benzofuran-3(2H)-one (compound 97);

5-[(6,7-dihydroxy-3-oxo-1-benzofuran-2(3H)-ylidene)methyl]-2,4(1H,3H)-pyrimidinedione (compound 98);

6,7-dihydroxy-2-[(1-methyl-1H-imidazol-2-yl)methylene]-1-benzofuran-3(2H)-one (compound 99);

6,7-dihydroxy-2-(1H-indol-7-ylmethylene)-1-benzofuran-3(2H)-one (compound 100);

6,7-dihydroxy-2-[(3-methyl-1-benzothien-2-yl)methylene]-1-benzofuran-3(2H)-one (compound 101);

2-(2,3-dihydro-1,4-benzodioxin-6-ylmethylene)-6,7-dihydroxy-1-benzofuran-3(2H)-one (compound 102);

2-(9-anthrylmethylene)-6,7-dihydroxy-1-ben-zofuran-3(2H)-one (compound 103);

6,7-dihydroxy-2-(1-pyrenylmethylene)-1-benzofuran-3(2H)-one (compound 104);

{5-[(6,7-dihydroxy-3-oxo-benzofuran-2(3H)-ylidene)methyl]-2-furyl}methyl acetate (compound 105);

6,7-dihydroxy-2-(9-phenanthrylmethylene)-1-benzofuran-3(2H)-one (compound 106);

2-(9H-fluoren-2-ylmethylene)-6,7-dihydroxy-1-benzofuran-3(2H)-one (compound 107);

2-[(10-chloro-9-anthryl)methylene]-6,7-dihydroxy-1-benzofuran-3(2H)-one (compound 108);

2-[(10-methyl-9-anthryl)methylene]-6,7-dihydroxy-1-benzofuran-3(2H)-one (compound 109);

6,7-dihydroxy-2-({5-[2-(trifluoromethyl)phenyl]-2-furyl}methylene)-1-benzofuran-3(2H)-one (compound 110);

2-{[5-(2-chlorophenyl)-2-furyl]methylene}-6,7-dihydroxy-1-benzofuran-3(2H)-one (compound 111);

2-[(4,5-dimethyl-2-furyl)methylene]-6,7-dihydroxy-1-benzofuran-3(2H)-one (compound 112);

2-[(5-bromo-2-furyl)methylene]-6,7-dihydroxy-1-benzofuran-3(2H)-one (compound 113);

2-{[5-(3-chlorophenyl)-2-furyl]methylene}-6,7-dihydroxy-1-benzofuran-3(2H)-one (compound 114);

6,7-dihydroxy-2-{[1-(phenylsulfonyl)-1H-pyrrol-2-yl]methylene}-1-benzofuran-3(2H)-one (compound 115);

6,7-dihydroxy-2-({5-[3-(trifluoromethyl)phenyl]-2-furyl}methylene)-1-benzofuran-3(2H)-one (compound 116);

2-[(5-ethyl-2-furyl)methylene]-6,7-dihydroxy-1-benzofuran-3(2H)-one (compound 117);

6,7-dihydroxy-2-[(5-chloro-2-thienyl)methylene]-1-benzofuran-3(2H)-one (compound 118);

2-[(5-chloro-3-methyl-1-phenyl-1H-pyrazol-4-yl)methylene]-6,7-dihydroxy-1-benzofuran-3(2H)-one (compound 119);

2-[(2,4-dimethoxy-5-pyrimidinyl)methylene]-6,7-dihydroxy-1-benzofuran-3(2H)-one (compound 120);

Group 3

6,7-dihydroxy-2-3-(4-hydroxy-3-methoxyphenyl)-2-propenylidene]-1-benzofuran-3(2H)-one (compound 121);

2-(3,3-diphenyl-2-propenylidene)-6,7-dihydroxy-1-benzofuran-3(2H)-one (compound 122);

6,7-dihydroxy-2-[2-methyl-3-phenyl-2-propenylidene]-1-benzofuran-3(2H)-one (compound 123);

2-{3-[4-(dimethylamino)phenyl]-2-propenylidene}-6,7-dihydroxy-1-benzofuran-3(2H)-one (compound 124); and

2-[3-(4-tert-butylphenyl)-2-methyl-2-propenylidene]-6,7-dihydroxy-1-benzofuran-3(2H)-one (compound 125).

### EXAMPLE 3

### 2-[1-(3,4-dimethoxyphenyl)ethylidene]-6,7-dihydroxy-1-benzofuran-3(2H)-one (compound 7)

To a solution of 6,7-dihydroxy-3(2H)-benzofuranone (20 mg, 0.12 mmol) and 3,4-dimethoxyacetophenone (22 mg, 0.12 mmol) in glacial acetic acid (1 mL), 37% HCl (0.04mL) was added and the solution stirred for 30 hours at room temperature. After removal of acetic acid under reduced pressure, the residue was purified by preparative HPLC on reverse phase column (Hypersil BDS 5 mcm, 250×10 mm.), eluant: (A)=water/acetonitrile/TFA 95:5:0.1; (B)=water/acetonitrile/TFA 5:95:0.1. Gradient: 0% (B) 3′, then 0-60% (B) 15′. Obtained pure title compound as yellow solid (36% yield). ¹H-NMR (400 Mhz, DMSOd₆), ppm: 2.65 (3H, s), 3.80 (6H, s), 6.7 (1H, d, J=8.2 Hz), 7.05 (2H, m), 7.35 (1H, m), 7.45 (1H, d, J=2 Hz), 7.47 (1H, m), 9.2 (1H, bs), 10.5 (1H, bs); MS m/z 329 [M+H]⁺.

By analogous procedure all the compounds of formula (I) with R₁=C₁-C₆ alkyl can be prepared:

6,7-dihydroxy-2-(1-phenylethylidene)-1-benzofuran-3(2H)-one (compound 126);

6,7-dihydroxy-2-[1-(4-hydroxyphenyl)ethylidene]-1-benzofuran-3(2H)-one (compound 127);

6,7-dihydroxy-2-[1-(3-hydroxyphenyl)ethylidene]-1-benzofuran-3(2H)-one (compound 128);

2-[1-(3,4-dihydroxyphenyl)ethylidene]-6,7-dihydroxy-1-benzofuran-3(2H)-one (compound 129);

2-[1-(2,4-dihydroxyphenyl)ethylidene]-6,7-dihydroxy-1-benzofuran-3(2H)-one (compound 130);

2-[1-(3-fluoro-4-hydroxyphenyl)ethylidene]-6,7-dihydroxy-1-benzofuran-3(2H)-one (compound 131);

2-[1-(3-hydroxy-4-fluorophenyl)ethylidene]-6,7-dihydroxy-1-benzofuran-3(2H)-one (compound 132).

### EXAMPLE 4

### 2-(3,4-dihydroxybenzyl)-6,7-dihydroxy-1-benzofuran-3(2H)-one (compound 89)

To a solution of 2-(3,4-dihydroxybenzylidene)-6,7-dihydroxy-1-benzofuran-3(2H)-one (200 mg, 0.7 mmol) in absolute ethanol (150 mL) 10% Pd on carbon (25 mg) is added and the reaction mixture is shaken under hydrogen (ca. 40 psi) into a Parr idrogenation apparatus at room temperature for 5 hours. After filtration of the catalyst and solvent evaporation the residue is purified by flash chromatography on silica gel (eluant: toluene/methanol 5:1). Obtained a white solid (48% yield). ¹H-NMR (400 Mhz, DMSOd₆), ppm: 2.7, 3.0 (2H, m), 4.83 (1H, m), 6.4-6.7 (4H, m), 6.9 (1H, d, J=8.3), 8.63 (1H, s), 8.69 (1H, s), 8.99 (1H, s), 10.29 (1H, s); MS m/z 289 [M+H]⁺.

By analogous procedure all the saturated compounds can be prepared:

2-benzyl-6,7-dihydroxy-1-benzofuran-3(2H)-one (compound 133);

6,7-dihydroxy-2-(4-hydroxybenzyl)-1-benzofuran-3(2H)-one (compound 134);

6,7-dihydroxy-2-(3-hydroxybenzyl)-1-benzofuran-3(2H)-one (compound 135);

2-(2,4-dihydroxybenzyl)-6,7-dihydroxy-1-benzofuran-3(2H)-one (compound 136);

2-[1-(3,4-dihydroxyphenyl)ethyl]-6,7-dihydroxy-1-benzofuran-3(2H)-one (compound 137);

6,7-dihydroxy-2-(3-pyridinylmethyl)-1-benzofuran-3(2H)-one (compound 138);

6,7-dihydroxy-2-[(6-methoxy-2-naphthyl)methyl]-1-benzofuran-3(2H)-one (compound 139);

6,7-dihydroxy-2-[(5-methoxy-1H-indol-2-yl)methyl]-1-benzofuran-3(2H)-one (compound 140);

6,7-dihydroxy-2-[(1-methyl-1H-benzimidazol-2-yl)methyl]-1-benzofuran-3(2H)-one (compound 141);

6,7-dihydroxy-2-[(1-methyl-1H-imidazol-2-yl)methyl]-1-benzofuran-3(2H)-one (compound 142);

6,7-dihydroxy-2-({5-[2-(trifluoromethyl)phenyl]-2-furyl}methyl)-1-benzofuran-3(2H)-one (compound 143).

The following example is provided for exemplification purposes only and is not intended to limit the scope of the invention described in broad terms above. All references cited in this disclosure are incorporated herein by reference.

### EXAMPLE 1

For humans, therapy with the disclosed compounds includes doses of a pharmaceutical formulation comprising one or more of the compounds of the invention that are from about 0.001 to about 100 mg/kg. Preferably, the dosage is about 0.1 to 10 mg/kg. The dosages will vary in accordance with, for example, the condition of the patient and the type of disease being treated. A dosage can be administered once or can be divided into a number of smaller doses to be administered at varying intervals of time. This therapy is effective in the treatment of telomerase-modulated diseases, including, for example, cancer related to abnormal cancer cell growth mediated by telomerase enzyme activity.

The invention and the manner and process of making and using it, are now described in such full, clear, concise and exact terms as to enable any person skilled in the art to which it pertains, to make and use the same. It is to be understood that the foregoing describes preferred embodiments of the present invention and that modifications can be made therein without departing from the spirit or scope of the present invention as set forth in the claims. To particularly point out and distinctly claim the subject matter regarded as invention, the following claims conclude this specification.

1. A method for inhibiting telomerase enzyme, which comprises contacting said enzyme with an effective amount of a compound having the following formula (I) or a pharmaceutically acceptable salt thereof, 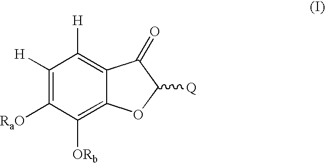
wherein
each of Rₐ and R_{b} represents, independently, hydrogen, C₁-C₆ alkyl, C₁-C₆ alkylcarbonyl or, Rₐ and R_{b}, taken together, represent methylene; [symbol]
Q represents a group of formula (a), (b), (c), (d) or (e) 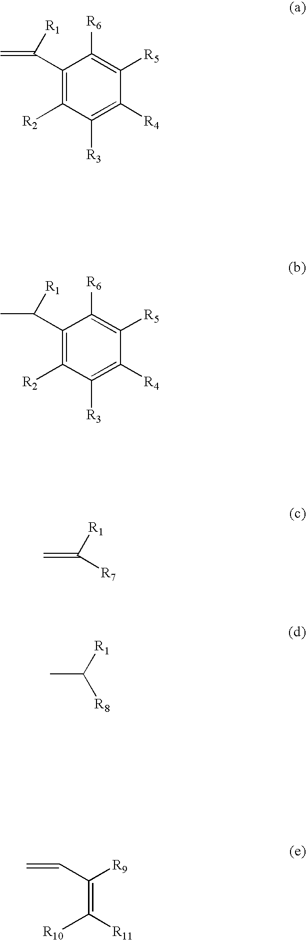
wherein
in a group of formula (a)
R₁ represents hydrogen or C₁-C₆ alkyl;
each of R₂, R₅ and R₆ represents, independently, hydrogen, halogen, hydroxy, C₁-C₆ alkyl, haloalkyl, C₁-C₆ alkenyl, C₁-C₆ alkoxy, C₁-C₆ alkenyloxy, aryloxy, arylalkoxy, haloalkoxy, C₁-C₆ alkoxycarbonyl, carboxyl, nitro or cyano; and
each of R₃ and R₄ represents, independently, hydrogen, halogen, hydroxy, C₁-C₆ alkyl, haloalkyl, optionally substituted alkenyl, optionally substituted arylalkenyl, optionally substituted alkynyl, optionally substituted arylalkynyl, aryl, C₁-C₆ alkoxy, aryloxy, arylalkoxy, haloalkoxy, aminoalkoxy, carbalkoxy, C₁-C₆ alkoxycarbonylalkoxy, carboxyl, C₁-C₆ alkoxycarbonyl, acyloxy, amino, dialkylamino, optionally substituted dialkylamino, acylamino, thioalkyl, arylsulfonyl, alkylsulfonyl, arylsulfenyl, alkylsulfenyl, arylsulfanyl, alkylsulfanyl, nitro or cyano, or R₃ and R₄ taken together represent methylenedioxy;
in a group of formula (b)
R₁ represents hydrogen or C₁-C₆ alkyl;
each of R₂, R₅ and R₆ represents, independently, hydrogen, halogen, hydroxy, C₁-C₆ alkyl, haloalkyl, C₁-C₆ alkoxy, aryloxy, C₁-C₆ alkoxycarbonyl, carboxyl or cyano; and
each of R₃ and R₄ represents, independently, hydrogen, halogen, hydroxy, C₁-C₆ alkyl, aryl, C₁-C₆ alkoxy, aryloxy, aminoalkoxy, carbalkoxy, C₁-C₆ alkoxycarbonylalkoxy, carboxylic acid, C₁-C₆ alkoxycarbonyl, acyloxy, amino, dialkylamino, optionally substituted dialkylamino, acylamino or cyano, or R₃ and R₄, taken together, represent methylenedioxy;
in a group of formula (c)
R₁ represents hydrogen; and
R₇ represents a fused polycyclic optionally substituted aryl or a monocyclic, bicyclic or tricyclic optionally substituted heteroaryl;
in a group of formula (d)
R₁ represents hydrogen; and
R₈ represents a fused polycyclic optionally substituted aryl or a monocyclic, bicyclic or tricyclic optionally substituted heteroaryl; and
in a group of formula (e)
R₉ represents hydrogen, C₁-C₆ alkyl, halogen or optionally substituted aryl;
R₁₀ represents C₁-C₆ alkyl, C₁-C₄ alkoxy, carboxyl, alkoxycarbonyl, optionally substituted aryl or optionally substituted heteroaryl; and
R₁₁ represents hydrogen, halogen or optionally substituted aryl.

2. A method for treating a telomerase-modulated disease, which comprises administering to a mammal a therapeutic effective amount of the compound of claim 1 or a pharmaceutically acceptable salt thereof.

3. A method for treating a cancer disease related to abnormal cancer cell growth mediated by telomerase enzyme activity, which comprises administering to a mammal a therapeutic effective amount of the compound of claim 1 or a pharmaceutically acceptable salt thereof.

4. A method for treating a cancer, which comprises administering to a mammal a therapeutic effective amount of the compound of claim 1 or a pharmaceutically acceptable salt thereof.

5. A pharmaceutical formulation for treating a telomerase-modulated disease, which comprises the compound of claim 1 or a pharmaceutically acceptable salt thereof and a pharmaceutically acceptable excipient.

6. A pharmaceutical formulation for treating a cancer disease related to abnormal cancer cell growth mediated by telomerase enzyme activity, which comprises the compound of claim 1 or a pharmaceutically acceptable salt thereof and a pharmaceutically acceptable excipient.

7. A pharmaceutical formulation for treating a cancer, which comprises the compound of claim 1 or a pharmaceutically acceptable salt thereof and a pharmaceutically acceptable excipient.

8. A compound of formula (IA) or a pharmaceutically acceptable salt thereof 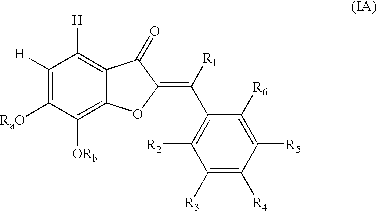
wherein
each of Rₐ and R_{b} represents, independently, hydrogen, C₁-C₆ alkyl, C₁-C₆ alkylcarbonyl or, Rₐ and R_{b}, taken together, represent methylene; and
R₁ represents hydrogen or C₁-C₆ alkyl;
each of R₂, R₅ and R₆ represents, independently, hydrogen, halogen, hydroxy, C₁-C₆ alkyl, haloalkyl, C₁-C₆ alkenyl, C₁-C₆ alkoxy, C₁-C₆ alkenyloxy, aryloxy, arylalkoxy, haloalkoxy, C₁-C₆ alkoxycarbonyl, carboxyl, nitro or cyano; and
each of R₃ and R₄ represents, independently, hydrogen, halogen, hydroxy, C₁-C₆ alkyl, haloalkyl, optionally substituted alkenyl, optionally substituted arylalkenyl, optionally substituted alkynyl, optionally substituted arylalkynyl, aryl, C₁-C₆ alkoxy, aryloxy, arylalkoxy, haloalkoxy, aminoalkoxy, carbalkoxy, C₁-C₆ alkoxycarbonylalkoxy, carboxyl, C₁-C₆ alkoxycarbonyl, acyloxy, amino, dialkylamino, optionally substituted dialkylamino, acylamino, thioalkyl, arylsulfonyl, alkylsulfonyl, arylsulfenyl, alkylsulfenyl, arylsulfanyl, alkylsulfanyl, nitro or cyano, or R₃ and R₄ taken together represent methylenedioxy; provided that:
(i) when R₁ is hydrogen and Rₐ and R_{b} are at the same time methyl, then R₂, R₃, R₄, R₅, and R₆ are not at the same time hydrogen;
(ii) when R₁ is hydrogen, Rₐ and R_{b} are at the same time methyl, R₂, R₄, R₅ and R₆ are at the same time hydrogen, then R₃ is different from NO₂;
(iii) when R₁ is hydrogen, Rₐ and R_{b} are at the same time methyl, R₂, R₃, R₅ and R₆ are at the same time hydrogen, then R₄ is different from methoxy;
(iv) when R₁ is hydrogen, Rₐ and R_{b} are at the same time methyl, R₂, R₅ and R₆ are at the same time hydrogen, then R₃, R₄ are not at the same time methoxy or R₃ and R₄ taken together are not methyienedioxy;
(v) R₁, R₂, R₃, R₄, R₅, R₆, Rₐ and R_{b} are not at the same time hydrogen;
(vi) when R₁, Rₐ, R_{b}, R₃, R₄, R₅ and R₆ are at the same time hydrogen, then R₂ is different from Cl, NO₂ or OH;
(vii) when R₁, Rₐ, R_{b}, R₂, R₄, R₅ and R₆ are at the same time hydrogen, then R₃ is different from Cl, NO₂ or OH;
(viii) when R₁, Rₐ, R_{b}, R₂, R₃, R₅ and R₆ are at the same time hydrogen, then R₄ is different from Cl, NO₂ or OH;
(ix) when R₁, Rₐ, R_{b}, R₂, R₅, and R₆ are at the same time hydrogen, then R₃ and R₄ are not at the same time methoxy or OH; or R₃ and R₄ taken together are not methylenedioxy;
(x) when R₁, Rₐ, R_{b}, R₂, R₅ and R₆ are at the same time hydrogen, then R₃ is different from OH and R₄ is different from methoxy;
(xi) when R₁, Rₐ, R_{b}, R₂, R₅ and R₆ are at the same time hydrogen, then R₃ is different from methoxy and R₄ is different from OH;
(xii) when R₁, Rₐ, R_{b}, R₃, R₅ and R₆ are at the same time hydrogen, then R₂ and R₄ are not at the same time OH;
(xiii) when R₁, Rₐ, R_{b}, R₂ and R₆ are at the same time hydrogen, then R₃, R₄ and R₅ are not at the same time OH or methoxy;
(xiv) when R₁, Rₐ, R_{b}, R₂ and R₆ are at the same time hydrogen, then R₄ is different from OH and R₃ and R₅ are not at the same time methoxy;
(xv) when R₁, R₂, R₅ and R₆ are at the same time hydrogen, and Rₐ and R_{b} are at the same time acetyl, then R₃ and R₄ are not at the same time acetyloxy;
(xvi) when R₁, R₂, and R₆ are at the same time hydrogen, then R₃, R₄ and R₅ are not at the same time methoxy and acetyloxy;
(xvii) when R₁, Rₐ, R₂, R₅ and R₆ are at the same time hydrogen and R_{b} is methyl, then R₃ and R₄ are not at the same time methoxy or OH; and
(xviii) when R₁, R₂, R₅ and R₆ are at the same time hydrogen, Rₐ is acetyl and R_{b} is methyl, then R₃ and R₄ are not at the same time methoxy.

9. A compound of formula (IB) or a pharmaceutically acceptable salt thereof 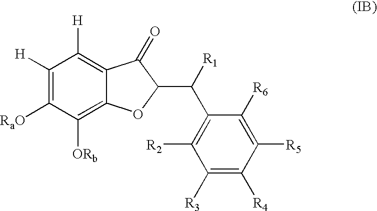
wherein
each of Rₐ and R_{b} represents, independently, hydrogen, C₁-C₆ alkyl, C₁-C₆ alkylcarbonyl or, Rₐ and R_{b}, taken together, represent methylene; and
R₁ represents hydrogen or C₁-C₆ alkyl;
each of R₂, R₅ and R₆ represents, independently, hydrogen, halogen, hydroxy, C₁-C₆ alkyl, haloalkyl, C₁-C₆ alkoxy, aryloxy, C₁-C₆ alkoxycarbonyl, carboxyl or cyano; and
each of R₃ and R₄ represents, independently, hydrogen, halogen, hydroxy, C₁-C₆ alkyl, aryl, C₁-C₆ alkoxy, aryloxy, aminoalkoxy, carbalkoxy, C₁-C₆ alkoxycarbonylalkoxy, carboxylic acid, C₁-C₆ alkoxycarbonyl, acyloxy, amino, dialkylamino, optionally substituted dialkylamino, acylamino or cyano, or R₃ and R₄, taken together, represent methylenedioxy.

10. A compound of formula (IC) or a pharmaceutically acceptable salt thereof 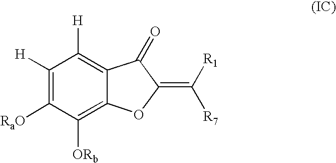
wherein
each of Rₐ and R_{b} represents, independently, hydrogen, C₁-C₆ alkyl, C₁-C₆ alkylcarbonyl or, Rₐ and R_{b}, taken together, represent methylene; and
R₁ represents hydrogen; and
R₇ represents a fused polycyclic optionally substituted aryl or a monocyclic, bicyclic or tricyclic optionally substituted heteroaryl; provided that:
(i) when R₁ is hydrogen and R₇ is a group of formula 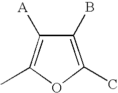
wherein A and B are at the same time hydrogen, then C is different from NO₂;
(ii) when R₁ is hydrogen and R₇ is a group of formula 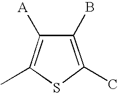
A,B and C are not at the same time hydrogen; and
(iii) when R₁ is hydrogen and R₇ is a group of formula 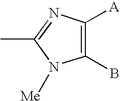
wherein A is hydrogen, then B is different from NO₂.

11. A compound of formula (ID) or a pharmaceutically acceptable salt thereof 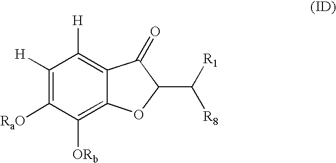
wherein
each of Rₐ and R_{b} represents, independently, hydrogen, C₁-C₆ alkyl, C₁-C₆ alkylcarbonyl or, Rₐ and R_{b}, taken together, represent methylene; and
R₁ represents hydrogen; and
R₈ represents a fused polycyclic optionally substituted aryl or a monocyclic, bicyclic or tricyclic optionally substituted heteroaryl.

12. A compound of formula (IE) or a pharmaceutically acceptable salt thereof 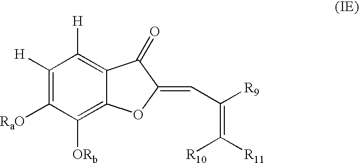
wherein
each of Rₐ and R_{b} represents, independently, hydrogen, C₁-C₆ alkyl, C₁-C₆ alkylcarbonyl or, Rₐ and R_{b}, taken together, represent methylene; and
R₉ represents hydrogen, C₁-C₆ alkyl, halogen or optionally substituted aryl;
R₁₀ represents C₁-C₆ alkyl, C₁-C₄ alkoxy, carboxyl, alkoxycarbonyl, optionally substituted aryl or optionally substituted heteroaryl; and
R₁₁ represents hydrogen, halogen or optionally substituted aryl;
provided that when R₉ and R₁₀ are at the same time hydrogen, then R₁₁ is different from unsubstituted phenyl.

13. A method for inhibiting a telomerase enzyme, which comprises contacting said enzyme with an effective amount of a compound selected from the group consisting of:
2-(3,4-dihydroxybenzylidene)-6,7-dihydroxy-1-benzofuran-3(2H)-one (compound 1);
2-(1,3-benzodioxol-5-ylmethylene)-6,7-dihydroxy-1-benzofuran-3(2H)-one (compound 2);
2-(3,4-dimethoxybenzylidene)-6,7-dihydroxy-1-benzofuran-3(2H)-one (compound 3);
2-(2,4-dihydroxybenzylidene)-6,7-dihydroxy-1-benzofuran-3(2H)-one (compound 4);
6,7-dimethoxy-2-(3-nitrobenzylidene)-1-benzofuran-3(2H)-one;
6,7-dimethoxy-2-(4-methoxybenzylidene)-1-benzofuran-3(2H)-one;
2-(1,3-benzodioxol-5-ylmethylene)-6,7-dimethoxy-1-benzofuran-3(2H)-one;
2-(3,4-dimethoxybenzylidene)-6,7-dimethoxy-1-benzofuran-3(2H)-one;
2-benzylidene-6,7-dihydroxy-1-benzofuran-3(2H)-one;
2-(4-chlorobenzylidene)-6,7-dihydroxy-1-benzofuran-3(2H)-one;
2-(3-chlorobenzylidene)-6,7-dihydroxy-1-benzofuran-3(2H)-one;
2-(2-chlorobenzylidene)-6,7-dihydroxy-1-benzofuran-3(2H)-one;
6,7-dihydroxy-2-(4-hydroxybenzylidene)-1-benzofuran-3(2H)-one;
6,7-dihydroxy-2-(3-hydroxybenzylidene)-1-benzofuran-3(2H)-one;
6,7-dihydroxy-2-(2-hydroxybenzylidene)-1-benzofuran-3(2H)-one;
6,7-dihydroxy-2-(4-nitrobenzylidene)-1-benzofuran-3(2H)-one;
6,7-dihydroxy-2-(3-nitrobenzylidene)-1-benzofuran-3(2H)-one;
6,7-dihydroxy-2-(2-nitrobenzylidene)-1-benzofuran-3(2H)-one;
6,7-dihydroxy-2-(3-hydroxy-4-methoxybenzylidene)-1-benzofuran-3(2H)-one;
6,7-dihydroxy-2-(4-hydroxy-3-methoxybenzylidene)-1-benzofuran-3(2H)-one;
6,7-dihydroxy-2-(3,4,5-trihydroxybenzylidene)-1-benzofuran-3(2H)-one;
6,7-dihydroxy-2-(3,4,5-trimethoxybenzylidene)-1-benzofuran-3(2H)-one;
6,7-dihydroxy-2-(4-hydroxy-3,5-dimethoxybenzylidene)-1-benzofuran-3(2H)-one;
6-(acetyloxy)-2-[3,4-bis(acetyloxy)benzylidene]-3-oxo-1-benzofuran-7(3H)-yl acetate;
6-(acetyloxy)-3-oxo-2-(3,4,5-trimethoxybenzylidene)-2,3-dihydro-1-benzofuran-7-yl acetate;
6-(acetyloxy)-3-oxo-2-[3,4,5-tris(acetyloxy)benzylidene]-2,3-dihydro-1-benzofuran-7-yl acetate;
2-(3,4-dimethoxybenzylidene)-6-hydroxy-7-methoxy-1-benzofuran-3(2H)-one;
2-(3,4-dihydroxybenzylidene)-6-hydroxy-7-methoxy-1-benzofuran-3(2H)-one;
2-(3,4-dimethoxybenzylidene)-7-methoxy-3-oxo-2,3-dihydro-1-benzofuran-6-yl acetate;
2-[1-(4-hydroxyphenyl)ethylidenel-6-methoxy-1-benzofuran-3(2H)-one;
4-[1-(6-methoxy-3-oxo-1-benzofuran-2(3H)-ylidene)ethyl)phenyl acetate;
2-[1-(3,4-dihydroxyphenyl)ethylidene]-4,6-dihydroxy-1-benzofuran-3(2H)-one;
6,7-dihydroxy-2-[(5-nitro-2-furyl)methylene]-1-benzofuran-3(2H)-one;
6,7-dimethoxy-2-[(5-nitro-2-furyl)methylene]-1-benzofuran-3(2H)-one;
6-(acetyloxy)-2-[(5-nitro-2-furyl)methylene]-3-oxo-2,3-dihydro-1-benzofuran-7-yl acetate;
6,7-dihydroxy-2-(2-thienylmethylene)-1-benzofuran-3(2H)-one;
6,7-dimethoxy-2-(2-thienylmethylene)-1-benzofuran-3(2H)-one;
6,7-dihydroxy-2-[(1-methyl-5-nitro-1H-imidazol-2-yl)methylenel-1-benzofuran-3(2H)-one;
6,7-dimethoxy-2-[(1-methyl-5-nitro-1H-imidazol-2-yl)methylene]-1-benzofuran-3(2H)-one;
6,7-dihydroxy-2-[3-phenyl-2-propenylidene]-1-benzofuran-3(2H)-one;
6-(acetyloxy)-3-oxo-2-[3-phenyl-2-propenylidene]-2,3-dihydro-1-benzofuran-7-yl acetate;
and a pharmaceutically acceptable salt thereof.

14. A compound selected from the group consisting of:
2-(3,4-dichlorobenzylidene)-6,7-dihydroxy-1-benzofuran-3(2H)-one (compound 5);
2-(3,4-dihydroxybenzylidene)-6,7-dimethoxy-1-benzofuran-3(2H)-one (compound 6);
2-[1-(3,4-dimethoxyphenyl)ethylidenel-6,7-dihydroxy-1-benzofuran-3(2H)-one (compound 7);
2-(2,5-dihydroxybenzylidene)-6,7-dihydroxy-1-benzofuran-3(2H)-one (compound 8);
2-(3-fluoro-2-hydroxybenzylidene)-6,7-dihydroxy-1-benzofuran-3(2H)-one (compound 9);
6,7-dihydroxy-2-[(7-methoxy-1,3-benzodioxol-5-yl)methylene]-1-benzofuran-3(2H)-one (compound 10);
6,7-dihydroxy-2-(2,4,6-trifluorobenzylidene)-1-benzofuran-3(2H)-one (compound 11);
6,7-dihydroxy-2-(2-hydroxy-3-methoxybenzylidene)-1-benzofuran-3(2H)-one (compound 12);
2-(3,5-dimethylbenzylidene)-6,7-dihydroxy-1-benzofuran-3(2H)-one (compound 13);
2-(3,4,5-trihydroxybenzylidene)-6,7-dihydroxy-1-benzofuran-3(2H)-one (compound 14);
2-(4-chloro-3-fluorobenzylidene)-6,7-dihydroxy-1-benzofuran-3(2H)-one (compound 15);
2-[4-(benzyloxy)benzylidene]-6,7-dihydroxy-1-benzofuran-3(2H)-one (compound 16);
5-(6,7-dihydroxy-3-oxo-1-benzofuran-2(3H)-ylidene)methyl]-2-hydroxybenzoic acid (compound 17);
2-(5-bromo-2-hydroxybenzylidene)-6,7-dihydroxy-1-benzofuran-3(2H)-one (compound 18);
3-[(6,7-dihydroxy-3-oxo-1-benzofuran-2(3H)-ylidene)methyl]benzoic acid (compound 19);
6,7-dihydroxy-2-[4-(phenylethynyl)benzylidene]-1-benzofuran-3(2H)-one (compound 20);
2-(3,5-ditert-butyl-2-hydroxybenzylidene)-6,7-dihydroxy-1-benzofuran-3(2H)-one (compound 21);
2-(3,5-dihydroxybenzylidene)-6,7-dihydroxy-1-benzofuran-3(2H)-one (compound 22);
3-{4-[(6,7-dihydroxy-3-oxo-1-benzofuran-2(3H)-ylidene)methyl]phenyl}-2-propenoic acid (compound 23);
2-(3,4-dihydroxy-5-methoxybenzylidene)-6,7-dihydroxy-1-benzofuran-3(2H)-one (compound 24);
2-[2-fluoro-4-(trifluoromethyl)benzylidene]-6,7-dihydroxy-1-benzofuran-3(2H)-one (compound 25);
6,7-dihydroxy-2-(3,4-dimethylbenzylidene)-1-benzofuran-3(2H)-one (compound 26);
2-[3-fluoro-4-(trifluoromethyl)benzylidene]-6,7-dihydroxy-1-benzofuran-3(2H)-one (compound 27);
2-(3-bromo-5-chloro-2-hydroxybenzylidene)-6,7-dihydroxy-1-benzofuran-3(2H)-one (compound 28);
2-[4-(dimethylamino)-2-methoxybenzylidene]-6,7-dihydroxy-1-benzofuran-3(2H)-one (compound 29);
2-[4-(benzyloxy)-2-hydroxybenzylidene)-6,7-dihydroxy-1-benzofuran-3(2H)-one (compound 30);
2-[4-(benzyloxy)-2-methoxybenzylidene]-6,7-dihydroxy-1-benzofuran-3(2H)-one (compound 31);
2-(2-fluoro-4-chlorobenzylidene)-6,7-dihydroxy-1-benzofuran-3(2H)-one (compound 32);
2-[2-(difluoromethoxy)benzylidene]-6,7-dihydroxy-1-benzofuran-3(2H)-one (compound 33);
6,7-dihydroxy-2-(2-vinylbenzylidene)-1-benzofuran-3(2H)-one (compound 34);
methyl2-[(6,7-dihydroxy-3-oxo-1-benzofuran-2(3H)-ylidene)methyl]-3,5-dimethoxybenzoate (compound 35);
2-[(6,7-dihydroxy-3-oxo-1-benzofuran-2(3H)-ylidene)methyl]benzonitrile (compound 36);
2-(2,3-dichlorobenzylidene)-6,7-dihydroxy-1-benzofuran-3(2H)-one (compound 37);
2-[4-(diethylamino)benzylidene]-6,7-dihydroxy-1-benzofuran-3(2H)-one (compound 38);
2-(2,4-dimethoxy-3-methylbenzylidene)-6,7-dihydroxy-1-benzofuran-3(2H)-one (compound 39);
6,7-dihydroxy-2-(2,3,4,5,6-pentamethylbenzylidene)-1-benzofuran-3(2H)-one (compound 40);
2-(2-bromo-4,5-dimethoxybenzylidene)-6,7-dihydroxy-1-benzofuran-3(2H)-one (compound 41);
2-(3,5-dimethoxybenzylidene)-6,7-dihydroxy-1-benzofuran-3(2H)-one (compound 42);
4-[(6,7-dihydroxy-3-oxo-1-benzofuran-2(3H)-ylidene)methyl]-2,6-dimethoxyphenyl acetate (compound 43);
2-(3-ethoxy-4-methoxybenzylidene)-6,7-dihydroxy-benzofuran-3(2H)-one (compound 44);
2-(2,4-difluorobenzylidene)-6,7-dihydroxy-1-benzofuran-3(2H)-one (compound 45);
2-(2,5-difluorobenzylidene)-6,7-dihydroxy-1-benzofuran-3(2H)-one (compound 46);
2-(2,6-difluorobenzylidene)-6,7-dihydroxy-1-benzofuran-3(2H)-one (compound 47);
2-(4-butoxybenzylidene)-6,7-dihydroxy-1-benzofuran-3(2H)-one (compound 48);
2-(3-chloro-4-fluorobenzylidene)-6,7-dihydroxy-1-benzofuran-3(2H)-one (compound 49);
2-(2,3,6-trichlorobenzylidene)-6,7-dihydroxy-1-benzofuran-3(2H)-one (compound 50);
2-(3,5-difluorobenzylidene)-6,7-dihydroxy-1-benzofuran-3(2H)-one (compound 51);
2-(2,3-difluorobenzylidene)-6,7-dihydroxy-1-benzofuran-3(2H)-one (compound 52);
2-(2,3,5-trichlorobenzylidene)-6,7-dihydroxy-1-benzofuran-3(2H)-one (compound 53);
2-(5-bromo-2,4-dimethoxybenzylidene)-6,7-dihydroxy-1-benzofuran-3(2H)-one (compound 54);
2-(2,6-dimethoxybenzylidene)-6,7-dihydroxy-1-benzofuran-3(2H)-one (compound 55);
2-4-(hexyloxy)benzylidene]-6,7-dihydroxy-1-benzofuran-3(2H)-one (compound 56);
2-(3-methyl-4-methoxybenzylidene)-6,7-dihydroxy-1-benzofuran-3(2H)-one (compound 57);
4-[(6,7-dihydroxy-3-oxo-1-benzofuran-2(3H)-ylidene)methyl]phenyl acetate (compound 58);
6,7-dihydroxy-2-(4-propoxybenzylidene)-1-benzofuran-3(2H)-one (compound 59);
2-(1,3-benzodioxol-4-ylmethylene)-6,7-dihydroxy-1-benzofuran-3(2H)-one (compound 60);
6,7-dihydroxy-2-(4-phenoxybenzylidene)-1-benzofuran-3(2H)-one (compound 61);
2-4-(benzyloxy)-3-methoxybenzylidene]-6,7-dihydroxy-1-benzofuran-3(2H)-one (compound 62);
2-(2-chloro-6-fluorobenzylidene)-6,7-dihydroxy-1-benzofuran-3(2H)-one (compound 63);
2-(2,3-dimethyl-4-methoxybenzylidene)-6,7-dihydroxy-1-benzofuran-3(2H)-one (compound 64);
2-(2,5-dimethyl-4-methoxybenzylidene)-6,7-dihydroxy-1-benzofuran-3(2H)-one (compound 65);
6,7-dihydroxy-2-(2,3,4,5,6-pentafluorobenzylidene)-1-benzofuran-3(2H)-one (compound 66);
6,7-dihydroxy-2-(3-pheroxybenzylidene)-1-benzofuran-3(2H)-one (compound 67);
2-[3-(4-chlorophenoxy)benzylidene]-6,7-dihydroxy-1-benzofuran-3(2H)-one (compound 68);
6,7-dihydroxy-2-[3-(4-methoxyphenoxy)benzylidene]-1-benzofuran-3(2H)-one (compound 69);
6,7-dihydroxy-2-[3-(4-methylphenoxy)benzylidene]-1-benzofuran-3(2H)-one (compound 70);
2-{4-[3-(dimethylamino)propoxy]benzylidene}-6,7-dihydroxy-1-benzofuran-3(2H)-one (compound 71);
2-(2-fluoro-4-bromobenzylidene)-6,7-dihydroxy-1-benzofuran-3(2H)-one (compound 72);
2-(2,4-diethoxy-3-methylbenzylidene)-6,7-dihydroxy-1-benzofuran-3(2H)-one (compound 73);
2-[2-chloro-5-(trifluoromethyl)benzylidene]-6,7-dihydroxy-1-benzofuran-3(2H)-one (compound 74);
2-[4-fluoro-2-(trifluoromethyl)benzylidene]-6,7-dihydroxy-1-benzofuran-3(2H)-one (compound 75);
2-[2-fluoro-6-(trifluoromethyl)benzylidene]-6,7-dihydroxy-1-benzofuran-3(2H)-one (compound 76);
2-(4-tert-butylbenzylidene)-6,7-dihydroxy-1-benzofuran-3(2H)-one (compound 77);
6,7-dihydroxy-2-(2,3,5,6-tetrafluorobenzylidene)-1-benzofuran-3(2H)-one (compound 78);
6,7-dihydroxy-2-[4-(trifluoromethoxy)benzylidene]-1-benzofuran-3(2H)-one (compound 79);
2-[4-(dibutylamino)benzylidene]-6,7-dihydroxy-1-benzofuran-3(2H)-one (compound 80);
2-{4-[bis(2-cyanoethyl)anino]benzylidene}-6,7-dihydroxy-1-benzofuran-3(2H)-one (compound 81);
6,7-dihydroxy-2-[3-(trifluoromethoxy)benzylidene]-1-benzofuran-3(2H)-one (compound 82);
2-(2-chloro-4-fluorobenzylidene)-6,7-dihydroxy-1-benzofuran-3(2H)-one (compound 83);
2-(2-methyl-3-fluorobenzylidene)-6,7-dihydroxy-1-benzofuran-3(2H)-one (compound 84);
2-[2-fluoro-3-(trifluoromethyl)benzylidene]-6,7-dihydroxy-1-benzofuran-3(2H)-one (compound 85);
2-[4-(difluoromethoxy)benzylidene]-6,7-dihydroxy-1-benzofuran-3(2H)-one (compound 86);
2-[2,5-bis(trifluoromethyl)benzylidene]-6,7-dihydroxy-1-benzofuran-3(2H)-one (compound 87);
2-[4-fluoro-3-(trifluoromethyl)benzylidene]-6,7-dihydroxy-1-benzofuran-3(2H)-one (compound 88);
2-(3,4-dihydroxybenzyl)-6,7-dihydroxy-1-benzofuran-3(2H)-one (compound 89);
6,7-dihydroxy-2-(3-pyridinylmethylene)-1-benzofuran-3(2H)-one (compound 90);
6,7-dihydroxy-2-[(6-hydroxy-4H-chromen-3-yl)methylene]-1-benzofuran-3(2H)-one (compound 91);
6,7-dihydroxy-2-(6-methoxy-2-naphthyl)methylene]-1-benzofuran-3(2H)-one (compound 92);
6,7-dihydroxy-2-[(5-methyl-2-thienyl)methylene]-1-benzofuran-3(2H)-one (compound 93);
6,7-dihydroxy-2-[(5-methoxy-1H-indol-2-yl)methylene]-1-benzofuran-3(2H)-one (compound 94);
6,7-dihydroxy-2-[(1-methyl-1H-benzimidazol-2-yl)methylene]-1-benzofuran-3(2H)-one (compound 95);
2-(1-acetyl-1H-indol-3-yl)methylene]-6,7-dihydroxy-1-benzofuran-3(2H)-one (compound 96);
6,7-dihydroxy-2-[(4-methyl-1H-imidazol-5-yl)methylene]-1-benzofuran-3(2H)-one (compound 97);
5-[(6,7-dihydroxy-3-oxo-1-benzofuran-2(3H)-ylidene)methyl]-2,4(1H,3H)-pyrimidinedione (compound 98);
6,7-dihydroxy-2-[(1-methyl-1H-imidazol-2-yl)methylene)-1-benzofuran-3(2H)-one (compound 99);
6,7-dihydroxy-2-(1H-indol-7-ylmethylene)-1-benzofuran-3(2H)-one (compound 100);
6,7-dihydroxy-2-(3-methyl-1-benzothien-2-yl)methylene]-1-benzofuran-3(2H)-one (compound 101);
2-(2,3-dihydro-1,4-benzodioxin-6-ylmethylene)-6,7-dihydroxy-1-benzofuran-3(2H)-one (compound 102);
2-(9-anthrylmethylene)-6,7-dihydroxy-1-benzofuran-3(2H)-one (compound 103);
6,7-dihydroxy-2-(1-pyrenylmethylene)-1-benzofuran-3(2H)-one (compound 104);
{5-[(6,7-dihydroxy-3-oxo-1-benzofuran-2(3H)-ylidene)methyl]-2-furyl}methyl acetate (compound 105);
6,7-dihydroxy-2-(9-phenanthrylmethylene)-1-benzofuran-3(2H)-one (compound 106);
2-(9H-fluoren-2-ylmethylene)-6,7-dihydroxy-1-benzofuran-3(2H)-one (compound 107);
2-[(10-chloro-9-anthryl)methylene]-6,7-dihydroxy-1-benzofuran-3(2H)-one (compound 108);
2-[(10-methyl-9-anthryl)methylene]-6,7-dihydroxy-1-benzofuran-3(2H)-one (compound 109);
6,7-dihydroxy-2-({5-[2-(trifluoromethyl)phenyl]-2-furyl}methylene)-1-benzofuran-3(2H)-one (compound 110);
2-{[5-(2-chlorophenyl)-2-furyl]methylene}-6,7-dihydroxy-1-benzofuran-3(2H)-one (compound 111);
2-[(4,5-dimethyl-2-furyl)methylene]-6,7-dihydroxy-1-benzofuran-3(2H)-one (compound 112);
2-[(5-bromo-2-furyl)methylene]-6,7-dihydroxy-1-benzofuran-3(2H)-one (compound 113);
2-{[5-(3-chlorophenyl)-2-furyl]methylene}-6,7-dihydroxy-1-benzofuran-3(2H)-one (compound 114);
6,7-dihydroxy-2-{[1-(phenylsulfonyl)-1H-pyrrol-2-yl]methylene}-1-benzofuran-3(2H)-one (compound 115);
6,7-dihydroxy-2-({5-[3-(trifluoromethyl)phenyl]-2-furyl}methylene)-1-benzofuran-3(2H)-one (compound 116);
2-[(5-ethyl-2-furyl)methylene]-6,7-dihydroxy-1-benzofuran-3(2H)-one (compound 117);
6,7-dihydroxy-2-[(5-chloro-2-thienyl)methylene]-1-benzofuran-3(2H)-one (compound 118);
2-[(5-chloro-3-methyl-1-phenyl-1H-pyrazol-4-yl)methylene]-6,7-dihydroxy-1-benzofuran-3(2H)-one (compound 119);
2-[(2,4-dimethoxy-5-pyrimidinyl)methylene]-6,7-dihydroxy-1-benzofuran-3(2H)-one (compound 120);
6,7-dihydroxy-2-[3-(4-hydroxy-3-methoxyphenyl)-2-propenylidene]-1-benzofuran-3(2H)-one (compound 121);
2-(3,3-diphenyl-2-propenylidene)-6,7-dihydroxy-1-benzofuran-3(2H)-one (compound 122);
6,7-dihydroxy-2-[2-methyl-3-phenyl-2-propenylidene]-1-benzofuran-3(2H)-one (compound 123);
2-{3-[4-(dimethylamino)phenyl]-2-propenylidene}-6,7-dihydroxy-1-benzofuran-3(2H)-one (compound 124);
2-[3-(4-tert-butylphenyl)-2-methyl-2-propenylidene]-6,7-dihydroxy-1-benzofuran-3(2H)-one (compound 125); and a pharmaceutically acceptable salt thereof.

15. A library of two or more compounds of formula (I) or a pharmaceutically acceptable salt thereof, 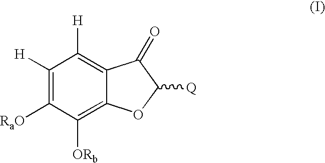
wherein
Rₐ and R_{b} represent hydrogen; [symbol]
Q represents a group of formula (a), (c) or (e) 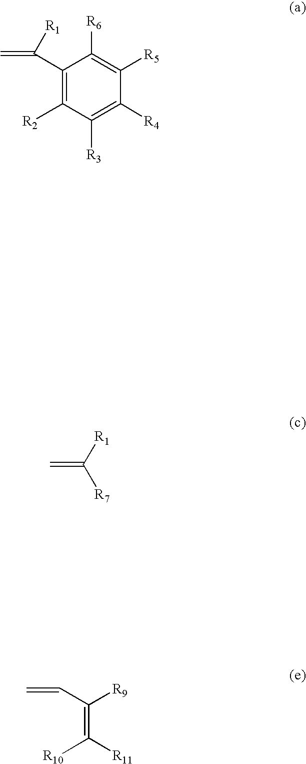
wherein
in a group of formula (a)
R₁ represents hydrogen;
each of R₂, R₅ and R₆ represents, independently, hydrogen, halogen, hydroxy, C₁-C₆ alkyl, haloalkyl, C₁-C₆ alkenyl, C₁-C₆ alkoxy, C₁-C₆ alkenyloxy, aryloxy, arylalkoxy, haloalkoxy, C₁-C₆ alkoxycarbonyl, carboxyl, nitro or cyano; and
each of R₃ and R₄ represents, independently, hydrogen, halogen, hydroxy, C₁-C₆ alkyl, haloalkyl, optionally substituted alkenyl, optionally substituted arylalkenyl, arylalkinyl, aryl, C₁-C₆ alkoxy, aryloxy, arylalkoxy, haloalkoxy, aminoalkoxy, carbalkoxy, C₁-C₆ alkoxycarbonylalkoxy, carboxyl, C₁-C₆ alkoxycarbonyl, acyloxy, amino, dialkylamino, optionally substituted dialkylamino, acylamino, thioalkyl, arylsulfonyl, alkylsulfonyl, arylsulfenyl, alkylsulfenyl, arylsulfanyl, alkylsulfanyl, nitro or cyano;
in a group of formula (c)
R₁ represents hydrogen; and
R₇ represents a fused polycyclic optionally substituted aryl or a monocyclic, bicyclic or tricyclic optionally substituted heteroaryl; and
in a group of formula (e)
R₉ represents hydrogen, C₁-C₆ alkyl, halogen or optionally substituted aryl;
R₁₀ represents C₁-C₆ alkyl, C₁-C₄ alkoxy, carboxyl, alkoxycarbonyl, optionally substituted aryl or optionally substituted heteroaryl and R₁₁ represents hydrogen, halogen or optionally substituted aryl.

16. The compound of claim 1 which produced by a combinatorial chemical process comprising reacting 6,7-dihydroxy-3(2H)-benzofuranone, with an aldehyde selected from the group consisting of:
2,5-dihydroxy benzaldehyde; 3-fluoro, 2-hydroxy benzaldehyde; 2,3-methylenedioxy benzaldehyde; 2,4,6-trifluoro benzaldehyde; 2-hydroxy, 3-methoxy benzaldehyde; 3,5-dimethyl benzaldehyde; 3,4,5-trihydroxy benzaldehyde; 4-chloro, 3-fluoro benzaldehyde; 4-(benzyloxy)benzaldehyde; 4-hydroxy, 3-carboxy benzaldehyde; 5-bromo, 2-hydroxy benzaldehyde; 3-carboxy benzaldehyde; 4-(phenylethynyl)benzaldehyde; 3,5-ditert-butyl, 2-hydroxy benzaldehyde; 3,5-dihydroxy benzaldehyde; 4-formylphenyl-2-propenoic acid; 3,4-dihydroxy, 5-methoxy benzaldehyde; 2-fluoro, 4-(trifluoromethyl) benzaldehyde; 3,4-dimethyl benzaldehyde; 3-fluoro-4-(trifluoromethyl)benzaldehyde; 3-bromo, 5-chloro,2-hydroxy benzaldehyde; 4-(dimethylamino), 2-methoxy benzaldehyde; 4-(benzyloxy), 2-hydroxy benzaldehyde; 4-(benzyloxy), 2-methoxy benzaldehyde; 2-fluoro, 4-chloro benzaldehyde; 2-(difluoromethoxy)benzaldehyde; 2-vinyl benzaldehyde; 2,4-dimethoxy, 6-methoxycarbonyl benzaldehyde; 2-cyano benzaldehyde; 2,3-dichloro benzaldehyde; 4-(diethylamino) benzaldehyde; 2,4-dimethoxy, 3-methyl benzaldehyde; 2,3,4,5,6 pentamethyl benzaldehyde; 2-bromo, 4,5-dimethoxy benzaldehyde; 3,5-dimethoxy benzaldehyde; 3,5-dimethoxy, 4-(acetoxy)benzaldehyde; 3-ethoxy, 4-methoxy benzaldehyde; 2,4-difluoro benzaldehyde; 2,5-difluoro benzaldehyde; 2,6-difluoro benzaldehyde; 4-butoxy benzaldehyde; 3-chloro, 4-fluoro benzaldehyde; 2,3,6-trichloro benzaldehyde; 3,5-difluoro benzaldehyde; 2,3-difluoro benzaldehyde; 2,3,5-trichloro benzaldehyde; 5-bromo, 2,4-dimethoxy benzaldehyde; 2,6-dimethoxy benzaldehyde; 4-hexyloxy benzaldehyde; 3-methyl, 4-methoxy benzaldehyde; 4-(acetoxy)benzaldehyde; 4-propoxy benzaldehyde; 2,3-methylenedioxy benzaldehyde; 4-phenoxy benzaldehyde; 4-(benzyloxy), 3-methoxy benzaldehyde; 2-chloro, 6-fluoro benzaldehyde; 2,3-dimethyl, 4-methoxy benzaldehyde; 2,5-dimethyl, 4-methoxy benzaldehyde; 2,3,4,5,6 pentafluoro benzaldehyde; 3-phenoxy benzaldehyde; 3-(4-chlorophenoxy)benzaldehyde; 3-(4-methoxyphenoxy)benzaldehyde; 3-(4-methylphenoxy)benzaldehyde; 4-(3-dimethylamino)propoxy benzaldehyde; 2-fluoro, 4-bromo benzaldehyde; 2,4-diethoxy, 3-methyl benzaldehyde; 2-chloro, 5-(trifluoromethyl)benzaldehyde; 4-fluoro, 2-(trifluoromethyl)benzaldehyde; 2-fluoro, 6-(trifluoromethyl)benzaldehyde; 4-tert-butyl benzaldehyde; 2,3,5,6-tetrafluoro benzaldehyde; 4-(trifluoromethoxy)benzaldehyde; 4-(dibutylamino)benzaldehyde; 4-[bis(2-cyanoethyl)amino benzaldehyde; 3-(trifluoromethoxy)benzaldehyde; 2-chloro, 4-fluoro benzaldehyde; 2-methyl, 3-fluoro benzaldehyde; 2-fluoro, 3-(trifluoromethyl)benzaldehyde; 4-(difluoromethoxy)benzaldehyde; 2,5-bis(trifluoromethyl)benzaldehyde; 4-fluoro, 3-(trifluoromethyl)benzaldehyde; 3-pyridinecarboxaldehyde; 6-hydroxychromene-3-carboxaldehyde; 6-methoxy-2-naphthaldehyde; 5-methyl-2-thiophenecarboxaldehyde; 5-methoxyindole-3-carboxaldehyde; 1-methyl-2-formylbenzimidazole; 4-hydroxy-3-methoxycinnamaldehyde; 3,3-diphenyl acrolein; alpha-methylcinnamaldehyde; 4-dimethylaminocinnamaldehyde; 1-acetyl-3-indolecarboxaldehyde; 5-methylimidazole-4-carboxaldehyde; 5-formyluracil; 1-methyl-2-imidazolecarboxaldehyde; 7-formylindole; 3-methylbenzo[b]thiophene-2-carboxaldehyde; 1,4-benzodioxan-6-carboxaldehyde; 9-anthraldehyde; 1-pyrenecarboxaldehyde; 5-acetoxymethyl-2-furaldehyde; phenanthrene-9-carboxaldehyde; 2-fluorenecarboxaldehyde; 10-chloro-9-anthraldehyde; 10-methylanthracene-9-carboxaldehyde; 5-[2-(trifluoromethyl)phenyl]furfural; 5-(2-chlorophenyl)furfural; 4,5-dimethyl-2-furancarboxaldehyde; 5-bromo-2-furaldehyde; 5-(3-chlorophenyl)-2-furaldehyde; 1-(phenylsulfonyl)-2-pyrrolecarboxaldehyde; 5-(3-trifluoromethylphenyl)furan-2-carboxaldehyde; 5-ethyl-2-furaldehyde; 5-chloro-2-thiophenecarboxaldehyde; 5-chloro-3-methyl-1-phenyl-1h-pyrazole-4-carbaldehyde; 5-formyl-2,4-dimethoxy-pyrimidine; and 3-(4-tert-butyl-phenyl)-2-methyl-propenal.

17. The compound of claim 1, for use in the preparation of a medicament having anticancer activity.

18. A pharmaceutical formulation which comprises the compound of claim 8 or a pharmaceutically acceptable salt thereof and a pharmaceutically acceptable excipient.

19. A pharmaceutical formulation which comprises the compound of claim 9.

20. A pharmaceutical formulation which comprises the compound of claim 10.

21. A pharmaceutical formulation which comprises the compound of claim 11.

22. A pharmaceutical formulation which comprises the compound of claim 12.

23. A combined anticancer therapy which comprises administering the compound of claim 1 or a pharmaceutically acceptable salt thereof with at least one other anticancer agent.

24. A product or kit comprising the compound of claim 1 or a pharmaceutical formulation of said compound and one or more anticancer agents, as a combined preparation for coordinated use in anticancer therapy.
